(19) United States
(12) Patent Application Publication
(10) Pub. No.: US 2004/0033213 A1
(43) Pub. Date: Feb. 19, 2004

(54) Methods of modulating dendritic cells using adjuvants

(75) Inventors: Edwin Walker, Portland, OR (US); C. Gregory Sowell, Mukilteo, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA

(21) Appl. No.: 10/377,995

(22) Filed: Feb. 28, 2003

Related U.S. Application Data
(60) Provisional application No. 60/360,641, filed on Feb. 28, 2002.

(51) Int. Cl. A61K 45/00; A61K 31/739; C12N 5/08

(52) U.S. Cl. 424/93.7; 514/54; 435/372

(57) ABSTRACT

The present invention relates to a method for culturing large amounts of antigen presenting cells, in particular of proliferating dendritic cell precursors, and of inducing their maturation ex vivo to mature dendritic cells. In addition, the present invention is directed to antigen-activated dendritic cells and provides methods of using such activated dendritic to elicit an immune response in a patient.

### CROSS-REFERENCES TO RELATED APPLICATIONS

The present application claims priority to U.S. Ser. No. 60/360,641, filed Feb. 28, 2002, herein incorporated by reference in its entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

Dendritic cells are one of the many types of cells that form the immune system. Dendritic cells are specialized in presenting antigens and initiating several T-dependent immune responses. Dendritic cells are distributed widely throughout the body in various tissues. They have been primarily classified by their tissue location and include interdigitating reticulum cells in lymphoid organs, veiled cells in afferent lymph, blood dendritic cells in the circulation, Langerhans cells in the epidermis, and dermal dendritic cells in the dermis of the skin (Steinman et al., Ann. Rev. Immunol. 9:271-296 (1991); and Steinman et al., J. Exp. Med. 137:1142-1162 (1973)). Dendritic cells are also found in non-lymphoid organs such as the heart, the lungs, the gut, and the synovium (Steinman (1991), supra).

As stated above, dendritic cells are potent antigen presenting cells (APCs) in the immune system and are critical for the initiation of primary immune responses. Accordingly, dendritic cells play an essential role in, for example, autoimmune diseases, graft rejection, human immunodeficiency virus infection, and the generation of T cell-dependent antibodies (Steinman, Annu. Rev. Immunol. 9:271-296 (1991)). Mature dendritic cells are also the principal stimulatory cells of primary mixed leukocyte reactions (Steinman et al, J. Exp. Med. 157:613 (1982); Kuntz Crow et al., Clin. Exp. Immunol. 49:338 (1986)).

Dendritic cells bind and modify antigens in a manner such that the modified antigen when presented on the surface of the dendritic cell can activate T-cells and B-cells. The modification of antigens by dendritic cells may, for example, include fragmenting a protein to produce peptides which have regions which specifically are capable of activating T-cells. The events whereby cells fragment antigens into peptides, and then present these peptides in association with products of the major histocompatibility complex (MHC) are termed “antigen presentation.”

The MHC is a region of highly polymorphic genes whose products are expressed on the surfaces of a variety of cells. MHC antigens are the principal determinants of graft rejection. Two different types of MHC gene products, class I and class II MHC molecules, have been identified. T cells recognize foreign antigens bound to only one specific class I or class II MHC molecule. The patterns of antigen association with class I or class II MHC molecules determine which T cells are stimulated. For instance, peptide fragments derived from extracellular proteins usually bind to class II MHC molecules, whereas proteins endogenously transcribed in dendritic cells generally associate with newly synthesized class I MHC molecules. As a consequence, exogenously and endogenously synthesized proteins are typically recognized by distinct T cell populations.

The ability to elicit an immune response in an individual has many important applications in both treating and preventing disease. Despite the successes achieved with the use of, e.g., vaccines, there are still many challenges in the field of vaccine development and immunotherapy. One such difficulty is the lack of immunogenicity of many antigens, i.e., the inability of an antigen to promote an effective immune response against the pathogen. In addition, certain antigens elicit only a certain type of immune response, for example, a cell-mediated or a humoral response.

The efficacy of dendritic cells in delivering antigens in such a way that a strong immune response ensues is widely acknowledged, but the use of these cells for immunotherapy has been hampered by the fact that there are very few in any given organ. In human blood, for example, only about 0.1% of the white cells are dendritic cells.

There is therefore a need in the field for methods to obtain mature dendritic cells in sufficient quantities to be clinically useful. The present invention fulfills this and other needs.

### SUMMARY OF THE INVENTION

In one aspect, the present invention provides a method of inducing maturation of dendritic cells ex vivo, the method comprising the steps of:

(i) incubating a culture of cells comprising dendritic cell progenitors with at least one cytokine or chemokine that promotes differentiation of dendritic cell progenitors;

(ii) pulsing the cells with antigen; and

(iii) stimulating the cells with a compound of Formula I below:

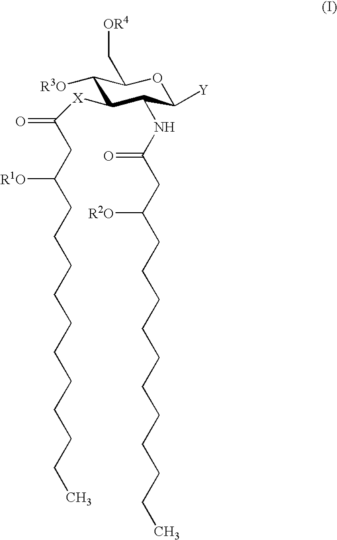

and pharmaceutically acceptable salts thereof, wherein X is —O— or —NH—; R¹ and R² are each independently a (C₂-C₂₄)acyl group, including saturated, unsaturated, substituted, unsubstituted, straight, and branched acyl groups; R³ is —H or —PO₃R¹²R¹³, wherein R¹² and R¹³ are each independently —H or (C₁-C₄)alkyl; R⁴ is —H, —CH₃ or —PO₃R¹⁴R¹⁵, wherein R¹⁴ and R¹⁵ are each independently selected from —H and (C₁-C₄)alkyl; and Y is a radical selected from the formulae:

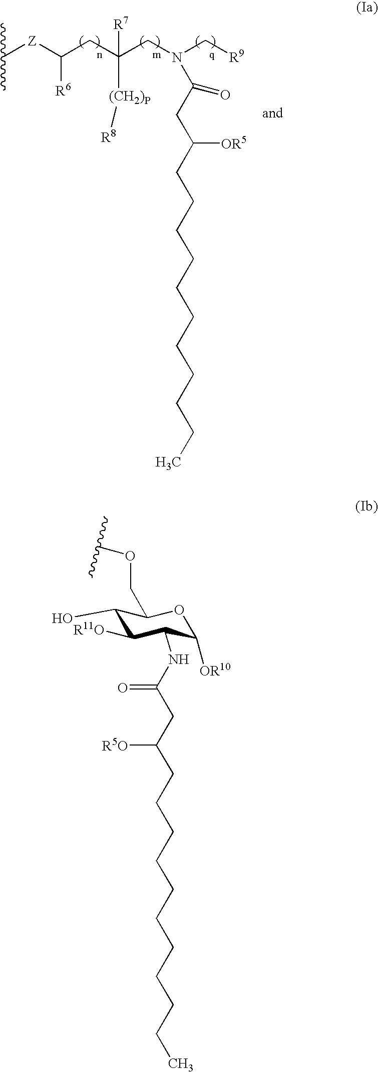

wherein the subscripts n, m, p and q are each independently an integer of from 0 to 6; R⁵ is H or a (C₂-C₂₄)acyl group including saturated, unsaturated, substituted, unsubstituted, straight, and branched acyl groups; R⁶ and R⁷ are independently selected from H and CH₃; R⁸ and R⁹ are independently selected from H, OH, (C₁-C₄)alkoxy, —PO₃H₂, —OPO₃H₂, —SO₃H, —OSO₃H, —NR¹⁶R¹⁷, —SR¹⁶, —CN, —NO₂, —CHO, —CO₂R¹⁶ and —CONR¹⁶R¹⁷, wherein R¹⁶ and R¹⁷ are each independently selected from H and (C₁-C₄)alkyl; R¹⁰ is selected from H, CH₃, —PO₃H₂, ω-phosphonoxy(C₂-C₂₄)alkyl, and ω-carboxy(C₁-C₂₄)alkyl; R¹¹ is H or a (C₂-C₂₄)acyl group including saturated, unsaturated, substituted, unsubstituted, straight, and branched acyl groups; and Z is —O— or —S—; with the proviso that when R³ is —PO₃R¹²R¹³, R⁴ is other than —PO₃R¹⁴R¹⁵;

thereby inducing maturation of dendritic cells.

In another embodiment, the compound is selected from the group consisting of the compounds disclosed in PCT 01/24284, filed Aug. 3, 2001; U.S. Pat. No. 6,525,028; and U.S. Ser. No. 10/068,398, filed Feb. 4, 2002, each herein incorporated by reference in their entirety. The present application is related to U.S. Ser. No. 60/220,081, filed Jul. 21, 2000, now abandoned, herein incorporated by reference in its entirety.

In another aspect, an immunogenically effective amount of the mature dendritic cells of the invention are administered to a mammal, preferably a human, thereby eliciting an immune response to the selected antigen.

In one embodiment, the cell is a human cell.

In one embodiment, the cytokine is selected from the group consisting of GM-CSF, IL-4, and TGF-β. In another embodiment, the GM-CSF is exogenously added to the culture. In another embodiment, the cells are incubated with exogenously added GM-CSF for 5-7 days or 10-12 days. In another embodiment, the cells are further incubated with TGF-β and/or IL-4.

In one embodiment, the cells are pulsed with antigen after stimulation of the cells with the compound of formula I. In another embodiment, the cells are pulsed with antigen before stimulation of the cells with the compound of formula I.

In one embodiment, the culture of cells is isolated from bone marrow, PBMC, CD14+ PBMC, or CD34+ PBMC.

In one embodiment, the cells are cryogenically stored.

In one embodiment, the antigen is selected from the group consisting of a cancer antigen, a viral antigen, a bacterial antigen, and a parasitic antigen. In another embodiment, the antigen is from a Mycobacterium sp., Chlamydia sp., Leishmania sp., Trypanosoma sp., Plasmodium sp., or a Candida sp., e.g., Mycobacterium tuberculosis or Trypanosoma cruzii.

In one embodiment, the antigen is associated with an autoimmune disorder.

In one embodiment, R₁═R₂═R₅═N—C₁₃H₂₇CO, X═Y═O, N=M=P=Q=0, R₆═R₇═R₈═R₉═R₄═H, and R₃═PO₃H₂. In another embodiment, R₁═R₂═R₅═N—C₁₁H₂₃CO, X═Y═O, N=M=Q=0, P=1, R₄═R₆═R₇═R₉═H, R₈═OH, and R₃═PO₃H₂. In another embodiment, R₁═R₂═R₅═N—C₉H₁₉CO, X═Y═O, N=M=Q=P=0, R₄═R₆═R₇═R₉═H, R₈═CO₂H, and R₃═PO₃H₂.

### BRIEF DESCRIPTION OF THE DRAWINGS

FIG. 1 shows the results from an IL-2 ELISA analysis of culture supernatants of DCs+D011.10 T cells. LPS stimulated the highest overall IL-2 secretion effect at all T cell:APC ratios tested, and this effect was higher than the response stimulated by DCs pre-stimulated with ova only. All the other DC pre-stimulation test groups (i.e., MPL®-S immunostimulant, RC-527, RC-529, and RC-544) were comparable in their ability to stimulate IL-2 secretion at all T cell:APC ratios.

FIG. 2 shows the effect of ova pulsed, 15 hour lipid A prestimulated DCs on IFN-γ secretion by D011.10 cells. As shown, RC-527 and LPS precultured DCs stimulate the highest levels of IFN-γ release by D011.10 T cells, and this effect is higher than that observed for ova only treated DCs. At 15 hours of preculture, the DCs stimulated by all the other-lipid A molecules shown are comparable (at all T cell:APC ratios) in their ability to stimulate IFN-γ secretion.

FIG. 3 shows that DCs precultured for 38 hours in the presence of lipid A molecules show a comparable effect to the 15 hour DC stimulation results in driving IFN-γ release by D011.10 T cells. All DC stimulation regimens produce DCs with significantly higher APC function than that observed for DCs pulsed with ova only.

FIG. 4 illustrates an effective T cell:APC ratio for the stimulation of IFN-γ secretion is 32:1. At this ratio the effect of LPS-stimulated, ova-pulsed DCs is 10-12 fold greater than the effect of DCs pulsed with ova only. As shown, the effect of DC prestimulation with RC-527 is comparable to that observed with LPS at this T cell:APC ratio, and MPL®-S immunostimulant and RC-529 preconditioning of DCs induce comparable stimulation of IFN-γ secretion.

FIG. 5 shows that lipid A precultured DCs induce the appearance of relatively high levels of the functionally active IL-12 (p70) when co-cultured with D011.10 T lymphocytes. LPS and RC-527 precultured DCs are most potent for this effect. All lipid A molecules are more effective in stimulating DC competence for this response than is ovalbumin alone.

FIG. 6 also shows that when lipid A precultured DCs are co-cultured with D011.10 T lymphocytes, an effective T cell:APC ratio for inducing the cytokine response is 16:1. Thus, RC-527 and LPS induce this DC-mediated IL-12 (p70) secretion response 12-14 times greater than the effect observed for ova only treated DCs. As shown, all the other lipid A molecules are most effective at this T cell:APC ratio.

FIG. 7 shows that IL-12 (p40) is released in the DC+D011.10 co-culture systems. LPS and RC-527 again have the most potent effect to stimulate the release of IL-12 (p40) homodimer. All lipid A molecules stimulate IL-12 (p40) responses which are greater than those observed for DCs precultured with antigen only.

FIG. 8 shows the results of an experiment in which DCs were grown in the presence of GM-CSF only, GM-CSF+5 ng/ml IL-4, GM-CSF+10 ng/ml IL-4 or GM-CSF+20 ng/ml IL-4, and were then tested at a 8:1 ratio of D011.10 effector T cells to DCs. LPS- (a) and MPL® immunostimulant- (b) activated DCs stimulate relatively comparable absolute levels of IFN-γ secretion for the three DC test groups. DCs cultured in GM-CSF only display a much higher ova only control background effect compared to the relatively uniform but much lower ova only control effect for DCs grown at all concentrations of IL-4. (c) shows the IFN-γ inductive effect of LPS- and MPL® immunostimulant-treated DCs (8:1 T cell to DC ratio) on all DC test populations relative to their respective ova only DC background controls. The LPS-induced IFN-γ effect for GM-CSF only DCs is comparable to that measured for DCs grown in GM-CSF plus the two highest doses of IL-4.

FIG. 9 shows the IL-2 secretory response (in pg/ml) of D011.10 T cells stimulated for 24 hours in the presence of ova-pulsed and LPS potentiated DCs (a) or in the presence of ova-pulsed and MPL® immunostimulant-activated DCs (b). The strength of the LPS- and MPL® immunostimulant -induced, antigen-specific, DC-mediated, IL-2 cytokine response for each DC test population is shown plotted as the % response above its corresponding ova-antigen-only DC background control (c).

FIG. 10 shows the effect of LPS stimulated, ova-pulsed, 10 day nonadherent DCs and DCs harvested from “mixed” induction cultures in triggering IFN-γ release from DC11.10 T cells at a ratio of 8:1 (T cells to DCs) (a). The ova only DC background control cells from 10 day GM-CSF cultures had a significantly lower background IFN-γ inductive effect. 10 day (GM-CSF only) LPS-stimulated, ova-pulsed DCs thus display a much higher % above the ova-only background IFN-γ effect (b).

FIG. 11 shows the induction of IL-2 expression by D011.10 cells subsequent to 24 hour in vitro stimulation by LPS-induced, ova-pulsed 10 day DCs precultured in GM-CSF only (a, b). The % IL-2 effect above the ova-only DC background values is higher than that seen for 6 day GM-CSF only cultured DCs.

FIG. 12 shows a set of correlated data for the IFN-γ inductive effects of 10 day GM-CSF only precultured DCs, pulsed with ova and potentiated overnight with LPS (a, b).

FIG. 13 illustrates the functional integrity of LPS- and RC-527-stimulated cryogenically stored, 10 day DCs in the D011.10T cell assay system. (a) shows the IFN-γ response induced by antigen-pulsed, lipid A stimulated DCs after 24 hours incubation with D011.10 T cells. (b) shows the IFN-γ response induced by LPS- and RC-527-stimulated, cryogenically stored, cultured DCs after 48 hours of incubation with D011.10 effector T cells. The 48 hours IFN-γ response triggered by LPS- and RC-527-stimulated, cryogenically stored DCs was comparable to the 24 hour IFN-γ response mediated by 10 day GM-CSF precultured DCs harvested directly from the expansion culture system prior to LPS and RC-527 stimulation (c).

FIG. 14 shows the results from a single experiment in which tetanus toxin pulsed (four hours), lipid A-stimulated (20 hours), seven-day cultured human DCs were tested for APC function against autologous, cryogenically stored T cells in a 6 day proliferation assay. (a) shows the CPM ³H-thymidine response for in vitro cultures of autologous T cells and stimulated DCs where the T cells:DC ratio was 30:1. All the DC test groups indicated stimulate significant levels of T cell proliferation above that observed for DCs pulsed with the “recall” tetanus antigen alone. (b) shows the stimulation index values for each of the test DC groups relative to the background tetanus antigen-only control DC test group.

FIG. 15 shows surface expression of CD80 in human dendritic cells stimulated with AGPs.

FIG. 16 shows surface expression of CD83 in human dendritic cells stimulated with AGPs.

FIG. 17 shows surface expression of CD40 in human dendritic cells stimulated with AGPs.

FIG. 18 shows surface expression of CD86 in human dendritic cells stimulated with AGPs.

### DESCRIPTION OF THE SPECIFIC EMBODIMENTS

I. Introduction

The present invention is based, at least in part, on the discovery that the addition of monophosphoryl lipid A, aminoalkyl glucosaminide phosphates, or derivatives thereof to a cell culture of antigen presenting cells (APCs) induces the maturation of the cells. Accordingly, the present invention provides methods for culturing large amounts of APCs and inducing their maturation ex vivo using such adjuvants.

In some aspects of the invention, the APCs cultured and matured as described herein are further pulsed with an antigen of interest. Such antigen-pulsed APCs are useful, for example, for immunotherapy. The present invention thus also provides methods for eliciting an immune response in a mammal by administering to the mammal APCs cultured and matured according to the disclosed methods, and presenting epitopes of an antigen of interest.

In a preferred embodiment of the invention, the APCs are dendritic cells, preferably from a human. The dendritic cells of the invention can be isolated, for example, from bone marrow, from PBMC, from CD14+ PBMC, or from CD34+ PBMC. The dendritic cells of the invention are preferably cultured for 5-7 days or for 10-12 days, in the presence of a cytokine or chemokine that promotes maturation of dendritic cells, e.g., G-CSF, IL-4, or TGF-β. In some embodiments, GM-CSF is exogenously added to the culture along with IL-4 and/or TGF-β, as well as other chemokines and cytokines.

In preferred embodiments, the maturation of the APCs of the invention is induced using monophosphoryl lipid A, aminoalkyl glucosaminide phosphates, or derivatives thereof, as shown in formula I below. In some aspects of the invention, the matured antigen presenting Cells of the invention can be stored cryogenically.

Where the antigen presenting cells are pulsed with an antigen, the antigen can be from any origin such as, for example, from a cancer, e.g., melanoma, renal cell carcinoma, breast cancer, pancreatic cancer, colorectal cancer, and testicular cancer; a virus, e.g., HCV, HBV, HIV, etc.; a bacterium, e.g., Mycobacterium sp.; a parasite, protozoa or fungus, e.g., Trypanosoma sp, Plasmodium sp., Chlamydia sp., Leishmania sp., or Candida sp; or an antigen associated with autoimmune disease, e.g., the variable region of an MHC molecule.

II. Definitions

“Antigen presenting cell progenitors” refers to cells that are capable of developing into a mature antigen presenting cell, e.g., a dendritic cell, or a macrophage. Antigen-presenting cell progenitors such as dendritic cell progenitors include, for example, bone marrow stem cells, monocytes, and partially differentiated cells such as CD14+ or CD34+ cells. Antigen presenting cell progenitors such as dendritic cell progenitors may be differentiated into mature cells by adding to the culture medium or stimulating the production of compounds, chemokines, and cytokines such as GM-CSF, in addition to IL-4, TGFβ, M-CSF, G-CSF, IL-3, IL-1, TNFα, CD40 ligand, LPS, flt3 ligand, SCF, FL, protein kinase C activators such as phorbol ester, and CD40 ligand, etc., and/or other compound(s), or combinations thereof, e.g., GM-CSF and IL-4; GM-CSF and TGFβ; GM-CSF, IL-4, and TGFβ; IL-3 and TNF; SCF and FL; IL-4 and TNF; FL and TNF; TNF and SCF; SCF, IL-1B, IL-3, IL-4, and IL-6; TGF-β and TNF; TGF-β and IL-4; GM-CSF, TNF and TGF-β in bovine serum free media, etc., that induce differentiation, maturation, and proliferation of antigen presenting cells (see, e.g., Paul, Fundamental Immunology (3 ed. 1993); see also Young, Curr. Opin. Hematol. 6:135-144 (1999); Agilette et al., Haematologica 83:824-848 (1998); and Gluckman et al., Cytokines, Cell. and Mol. Ther. 3:187-196 (1997)). “Dendritic cells” are highly potent APCs (Banchereau & Steinman, Nature 392:245-251 (1998) and have been shown to be effective as a physiological adjuvant for eliciting prophylactic or therapeutic antitumor immunity (see Timmerman & Levy, Ann. Rev. Med. 50:507-529 (1999)). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention.

Dendritic cells are conveniently categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for B and T cell activation such as class I and class II MHC molecules, adhesion molecules (e.g., CD54, CD 18, and CD11) and costimulatory molecules (e.g., CD40, CD80, CD83, CD86 and 4-1BB).

A “cytokine or chemokine that promotes differentiation of antigen presenting cell progenitors” refers to any cytokine or chemokine that drives stem cells or partially differentiated cells to a more mature or differentiated APC, e.g., dendritic cell, phenotype, e.g., a compound that drives a stem cell or a partially differentiated cell to an immature or mature dendritic cell phenotype. The cytokine can be provided exogenously to the cell culture, or can be provided by cells in the culture that express the cytokine or chemokine, either an endogenous or a recombinant protein. For expression of a recombinant protein, cells in the culture are transfected with an expression vector encoding the chemokine or cytokine, which then produces the protein.

“Antigen” refers to a peptide or polypeptide comprising one or more MHC class I or MHC class II epitopes. Thus, an antigen can be a protein or polypeptide, fragment of a protein or polypeptide, or a peptide comprising one or more epitopes. The antigen can be provided exogenously to the cell culture, or can be provided by cells in the culture the express the antigen, either an endogenous or recombinant protein. For expression of a recombinant protein, cells in the culture are transfected with an expression vector encoding the antigen, which then produces the protein. The antigen may be a whole protein or fragment thereof, or an MHC II epitope of about 8 to 25 amino acid residues, more preferably 9-15 amino acid residues. Dendritic cells of the invention can be pulsed with antigen either before or after administration of the adjuvant compound of formula I.

Monophosphoryl lipid A compounds (MPLs) refer to naturally occurring components of bacterial lipopolysaccharide (refined detoxified endotoxin), such as monophosphoryl lipid A, and derivatives thereof, such as 3-de-O-acylated monophosphoryl lipid A (3D-MPL). MPL adjuvants are available from Corixa Corporation (Seattle, Wash.; see U.S. Pat. Nos. 4,436,727; 4,436,728; 4,987,237; 4,877,611; 4,866,034 and 4,912,094 for structures and methods of isolation and synthesis). A structure of MPL is disclosed, e.g., in U.S. Pat. No. 4,987,237.

“Aminoalkyl glucosaminide phosphate” (AGP) compounds generally comprise a 2-deoxy-2-amino-α-D-glucopyranose (glucosaminide) in glycosidic linkage with an aminoalkyl (aglycon) group. Suitable AGP compounds, and methods for their synthesis and use, are described in U.S. patent application Ser. Nos. 08/853,826, 09/074,720 and 09/439,839, the disclosures of which are incorporated herein by reference in their entireties. See also Johnson et al., J. Med. Chem. 42:4640-4649 (1999); Johnson et al., Bioorganic & Medicinal Chemistry Letters 9:2273-2278 (1999); and WO98/50399 for methods of synthesis.

In general, the synthetic methods described in the above-noted references are broadly applicable to the preparation of compounds having different acyl groups and substitutions. One of skill in the art will appreciate that the convergent methods described therein can be modified to use alternate acylating agents, or can be initiated with commercially available materials having appropriate acyl groups attached.

The maturation inducing-compounds of the subject invention, including monophosphoryl lipid A compounds and AGP compounds, can be described generally by Formula I below:

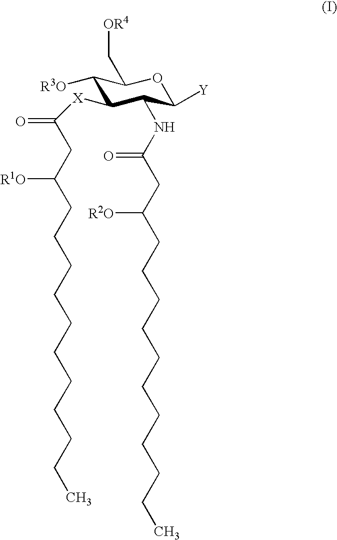

and pharmaceutically acceptable salts thereof, wherein X is —O— or —NH—; R¹ and R² are each independently a (C₂-C₂₄)acyl group, including saturated, unsaturated, substituted, unsubstituted, straight, and branched acyl groups; R³ is —H or —PO₃R¹²R¹³, wherein R¹² and R¹³ are each independently —H or (C₁-C₄)alkyl; R⁴ is —H, —CH₃ or —PO₃R¹⁴R¹⁵, wherein R¹⁴ and R¹⁵ are each independently selected from —H and (C₁-C₄)alkyl; and Y is a radical selected from the formulae:

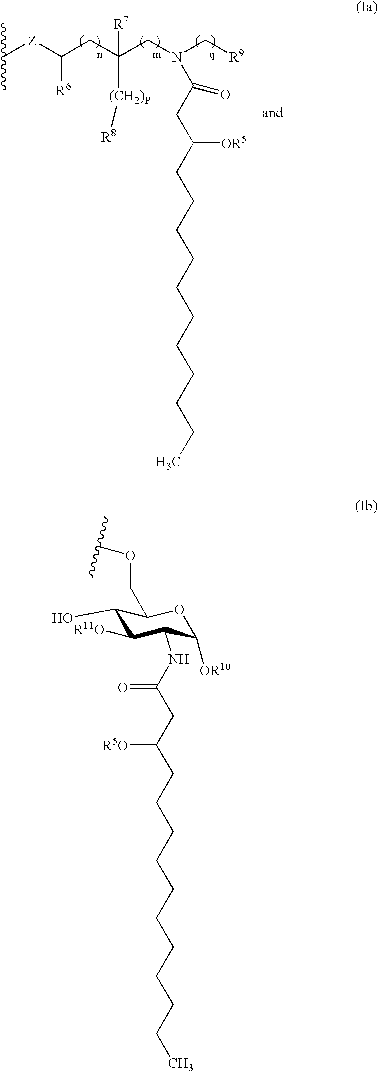

wherein the subscripts n, m, p and q are each independently an integer of from 0 to 6; R⁵ is H or a (C₂-C₂₄)acyl group including saturated, unsaturated, substituted, unsubstituted, straight, and branched acyl groups; R⁶ and R⁷ are independently selected from H and CH₃; R⁸ and R⁹ are independently selected from H, OH, (C₁-C₄)alkoxy, —PO₃H₂, —OPO₃H₂, —SO₃H, —OSO₃H, —NR¹⁶R¹⁷, —SR¹⁶, —CN, —NO₂, —CHO, —CO₂R¹⁶, and —CONR¹⁶R¹⁷, wherein R¹⁶ and R¹⁷ are each independently selected from H and (C₁-C₄)alkyl; R¹⁰ is selected from H, CH₃, —PO₃H₂, ω-phosphonoxy(C₂-C₂₄)alkyl, and ω-carboxy(C₁-C₂₄)alkyl; R¹¹ is H or a (C₂-C₂₄)acyl group including saturated, unsaturated, substituted, unsubstituted, straight, and branched acyl groups; and Z is —O— or —S—; with the proviso that when R³ is —PO₃R¹²R¹³, R⁴ is other than —PO₃R⁴R¹⁵.

In the general formula above, the configuration of the 3′ stereogenic centers to which the normal fatty acid acyl residues are attached is R or S, but preferably R. The stereochemistry of the carbon atoms to which R⁶ and R⁷ are attached can be R or S. All stereoisomers, enantiomers, diastereomers and mixtures thereof are considered to be within the scope of the present invention.

In one particularly preferred embodiment of the invention, the AGP is RC-529, which comprises a 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-β-D-glucopyranoside triethylammonium salt. This corresponds to a compound having the structure set forth in Formula I in which R₁═R₂═R₅=n-C₁₃H₂₇CO, X═Y═O, n=m=p=q=0, R₆═R₇═R₈═R₉═R₄═H, and R₃═PO₃H₂.

In additional embodiments of the invention, preferred AGP compounds of Formula I include the following:

| | Ref. | | | | | | |
|---|---|---|---|---|---|---|---|
| Compound | No. | R₁, R₂, R₅ | n | p | R₈ | q | R₉ |
| RC-517 | B2** | n-C₁₃H₂₇CO | 0 | 1 | OH | 0 | H |
| RC-544 | B3 | n-C₁₁H₂₃CO | 0 | 1 | OH | 0 | H |
| RC-540 | B9 | n-C₉H₁₉CO | 1 | 1 | OH | 0 | H |
| RC-547 | B14** | n-C₉H₁₉CO | 0 | 0 | CO₂H | 0 | H |
| RC-527 | B15* | n-C₉H₁₉CO | 0 | 0 | CO₂H | 0 | H |
| RC-525 | B21 | n-C₁₃H₂₇CO | 1 | 0 | H | 0 | H |
| RC-557 | B22 | n-C₁₃H₂₇CO | 2 | 0 | H | 0 | H |
| RC-522 | B25 | n-C₉H₁₉CO | 0 | 0 | CONH₂ | 0 | H |

For all compounds shown: X = Y = O; R₆ = R₇ = H; m = 0; R₃phosphono; R₄ = H.
*the stereochemistry of the carbon atom to which R₇ is attached is S.
**the stereochemistry of the carbon atom to which R₇ is attached is R.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons), the chain or cyclic radical optionally interrupted by a heteroatom such as oxygen, nitrogen, and sulfur. Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Typically, an alkyl group will have from 1 to 24 carbon atoms. A “lower alkyl” or is a shorter chain alkyl group, generally having eight or fewer carbon atoms.

“Substituted” includes “hydroxy substituted.”

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “acyl” refers to a group derived from an organic acid by removal of the hydroxy group. Examples of acyl groups include acetyl, propionyl, dodecanoyl, tetradecanoyl, isobutyryl, and the like. Accordingly, the term “acyl” is meant to include a group otherwise defined as —C(O)-alkyl.

Each of the above terms (e.g., “alkyl” “acyl”) are meant to include both substituted and unsubstituted forms of the indicated radical. Preferred substituents for each type of radical are provided below.

Substituents for the alkyl and acyl radicals can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′-C(O)NR″R′″, —NR′C(O)₂R′, —NH—C(NH₂)═NH, —NR′C(NH₂)═NH, —NHC(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen and unsubstituted (C₁-C₈)alkyl. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and the like.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, et al., Journal of Pharmaceutical Science, 66:1-19 (1977)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

III. Cells and Cell Culture

The present invention provides methods of culturing and inducing maturation of antigen presenting cells (APCs) ex vivo. Specifically, the present invention is directed to methods for culturing and inducing the maturation of dendritic cells (DCs). In addition, the present invention provides methods of pulsing the cultured, matured APCs with an antigen of interest.

A. Types of cells

Any antigen presenting cell (APC) can be used with the methods of the present invention. The term APC encompasses any cell capable of handling and presenting an antigen to lymphocytes. Typically, APCs include, e.g., macrophages, Langerhans dendritic cells and Follicular dendritic cells. In addition, B cells have also been shown to have an antigen presenting function and are thus contemplated by the present invention. In preferred embodiments of the present invention, the APCs are dendritic cells.

B. Source of cells

APCs can be isolated from any of the tissues where they reside and which are known to those of skill in the art. In particular, dendritic cells and their progenitors may be obtained from any tissue source comprising dendritic cell precursors that are capable of proliferating and maturing in vitro into dendritic cells, when cultured and induced to mature according to the methods of the present invention. Such suitable tissue sources include, e.g., peripheral blood, bone marrow, tumor-infiltrating cells, peritumoral tissues-infiltrating cells, lymph node biopsies, thymus, spleen, skin, umbilical cord blood, monocytes harvested from peripheral blood, CD34 or CD14 positive cells harvested from peripheral blood, blood marrow or any other suitable tissue or fluid. In the context of the present invention, dendritic cells are preferably isolated from bone marrow or from peripheral blood mononuclear cells (PBMCs).

Peripheral blood can be collected using any standard apheresis procedure known in the art (see, e.g., Bishop et al., Blood 83:610:616 (1994)). PBMCs can then be prepared from whole blood samples by separating mononuclear cells from red blood cells. There are a number of methods for isolating PBMCs including, e.g., velocity sedimentation, isopyknic sedimentation, affinity purification, and flow cytometry. Typically, PBMCs are separated from red blood cells by density gradient (isopyknic) centrifugation, in which the cells sediment to an equilibrium position in the solution equivalent to their own density. For density gradient centrifugation, physiological media should be used, the density of the solution should be high, and the media should exert little osmotic pressure. Density gradient centrifugation uses solutions such as sodium ditrizoate-polysucrose, Ficoll, dextran, and Percoll (see, e.g., Freshney, Culture of Animal Cells, 3rd ed. (1994)). Such solutions are commercially available, e.g., HISTOPAQUE® (Sigma). Examples of methods for isolating dendritic cells from PBMCs are disclosed in, e.g., U.S. Pat. Nos. 6,017,527 and 5,851,756; and in O'Doherty et al., J. Exp. Med. 178:1067-1078 (1993); Young and Steinman, J. Exp. Med. 171:1315-1332 (1990); Freudenthal and Steinman, Proc. Natl. Acad. Sci. USA 57:7698-7702 (1990); Macatonia et al., Immunol. 67: 285-289 (1989); and Markowicz and Engleman, J. Clin. Invest. 85:955-961 (1990).

CD34+ PBMCs or CD14+ PBMCs can further be selected as a preferred source of dendritic cells using a variety of selection techniques known to those of skill in the art. For example, monoclonal antibodies (or any protein-specific binding protein) can be used to bind to a cell surface antigen found on the surface of the PBMC sub-population of interest (e.g., CD34 or CD14 on the surface of CD34+ or CD 14+ PBMCs, respectively). Binding of such specific monoclonal antibodies allows the identification and isolation of the sub-group of PBMCs of interest from a total PBMC population by any of a number of immunoaffinity methods known to those of skill in the art. Examples of immunoaffinity methods for isolating sub-populations of PBMCs are described in, e.g., U.S. Pat. No. 6,017,527.

Alternatively, the dendritic cells of the present invention can be isolated from bone marrow. For a general description of methods for isolating dendritic cells from bone marrow see, e.g., U.S. Pat. No. 5,994,126; Dexter et al., in Long-Term Bone Marrow Culture, pages 57-96, Alan R. Liss, (1984); and Lutz et al., J. Immunol. Methods 223:77-92 (1999). Dendritic cells from bone marrow can typically be obtained from a number of different sources, including, for example, from aspirated marrow. Alternatively, bone marrow can be extracted from a sacrificed animal by dissecting out the femur, removing soft tissue from the bone and removing the bone marrow with a needle and syringe. Dendritic cells can be identified among the different cell types present in the bone marrow based on their morphological characteristics. For example, cultured immature dendritic cells in one or more phases of their development are loosely adherent to plastic, flattening out with a stellate shape.

In a preferred embodiment, the present invention provides methods to grow large numbers of murine dendritic cells from mouse bone marrow-derived dendritic cell progenitors. In another preferred embodiment, the present invention provides methods to grow large number of human dendritic cells obtained from CD14 positive human peripheral blood monocyte precursors.

Optionally, prior to culturing the cells, the tissue source can be pre-treated to remove cells that may compete with the proliferation and/or the survival of the dendritic cells or of their precursors. Examples of such pre-treatments are described, e.g., in U.S. Pat. No. 5,994,126.

C. Number of days

Those of skill in the art will recognize that APCs can be cultured for any suitable amount of time. Typically, APCs are cultured from 4 to 15 days. In a preferred embodiment, the APCs of the invention are cultured for 5-7 days (Inaba et al., J. Exp. Med. 176:1693 (1992); Inaba et al., J. Exp. Med. 175:1157 (1992); Inaba et al., Current Protocols Immunol., Unit 3.7 (Coico et al., eds. 1998); Schneider et al., J. Immunol. Meth. 154:253 (1992)). In another preferred embodiment, the APCs of the invention are cultured for 10-12 days (Lutz et al., supra).

D. Compounds added: adjuvants and growth factors

1. Cytokines and Chemokines

GM-CSF has been found to promote the proliferation in vitro of both nonadherent immature dendritic cells and adherent macrophages (see, e.g., U.S. Pat. No. 5,994,126; and Lutz et al., supra). In the context of the present invention, precursor dendritic cells are thus preferably cultured in the presence of GM-CSF at a concentration sufficient to promote their survival and proliferation. The dose of GM-CSF depends, e.g., on the amount of competition from other cells (especially macrophages and granulocytes) for the GM-CSF, and on the presence of GM-CSF inactivators in the cell population (see, e.g., U.S. Pat. No. 5,994,126). The GM-CSF concentration is typically of about 1 ng/ml to 100 ng/ml, preferably of about 5 ng/ml to about 20 ng/ml. GM-CSF can be obtained from different sources well known to those of skill in the art (see, e.g., Lutz et al., supra; and U.S. Pat. No. 5,994,126).

In addition to GM-SCF, a variety of cytokines have been shown to induce the proliferation and/or maturation of dendritic cells and other APCs, and are suitable for use with the methods of the present invention (see, e.g., Caux et al., J. Exp. Med. 180:1263-1272 (1984); Allison, Archivum Immunologiae et Therapiae Experimentalis 45:141-147 (1997)). Cytokines that can be used to enhance the maturation of dendritic cells ex vivo include, but are not limited to, TNF-alpha, stem cell factor (SCF; also named c-kit ligand, steel factor (SF), mast cell growth factor (MGF); see, e.g., EP 423,980; and U.S. Pat. No. 6,017,527), granulocyte colony-stimulating factor (G-CSF), monocyte-macrophage colony-stimulating factor (M-CSF), , as well as a number of interleukins, such as, e.g., IL-1α and IL-1β, IL-3, IL-4, IL-6, and IL-13 (see, e.g., U.S. Pat. No. 6,017,527 and 5,994,126). In addition to promoting the maturation of dendritic cells, some interleukins (e.g., IL-4) have been shown to suppresses the overall growth of macrophages and thus favors higher levels of pure DC growth. Cytokines are used in amounts which are effective in increasing the proportion of dendritic cells present in the culture by enhancing either the proliferation or the survival of dendritic cell precursors.

In preferred embodiments, the dendritic cell precursors of the present invention are cultured in the presence of GM-CSF. In other preferred embodiments, the dendritic cells of the present invention are cultured in the presence of both GM-CSF and IL-4. When human dendritic precursor cells are cultured, the GM-CSF is preferably human GM-CSF (huGM-CSF).

2. Adjuvants

The present invention is further based, at least in part, on the discovery that a variety of adjuvants can be used to stimulate the maturation ex vivo of immature dendritic cells cultured as described above. Specifically, immature dendritic cells can be harvested from the induction cultures described supra and their maturation to end-stage antigen presenting cells can be induced by treating the cells with a variety of adjuvants. Adjuvants that promote the maturation of dendritic cells include, but are not limited to, MPL® immunostimulant and selected synthetic lipid A analogs such as aminoalkyl glucosamide phosphate (AGP). Synthetic lipid A analogs include, for example, lipid A monosaccharide synthetics such as RC-529, RC-544 and RC-527, and the disaccharide mimetic, RC-511. These adjuvants are typically used as 10% ethanol-in-water formulations, although any other formulation that promotes the maturation of dendritic cells is suitable for use with the methods of the present invention. Adjuvants that can be used with the methods of the present invention can be synthesized or obtained from a variety of sources (see, e.g., Lutz et al., supra; Johnson et al., Bioorganic Medicinal Chemistry Letters 9:2273-2278 (1999)).

In preferred embodiments, the maturation of dendritic cells to end-stage APCs is induced using MPL or AGP.

E. Description of the maturation of DCs

The maturation of DCs can be followed using a number of molecular markers and of cell surface phenotypic alterations. These changes can be analyzed, for example, using flow cytometry techniques. Typically, the maturation markers are labeled using specific antibodies and DCs expressing a marker or a set of markers of interest can be separated from the total DC population using, for example, cell sorting FACS analysis. Markers of DC maturation include genes that are expressed at higher levels in mature DCs compared to immature DCs. Such markers include, but are not limited to, cell surface MHC Class II antigens (in particular HLA-DR), ICAM-1, B7-2, costimulating molecules such as CD 40, CD 80, CD 86, CD 83, cell trafficking molecules such as CD 54, CD 11b and CD 18, etc. Furthermore, mature dendritic cell can be identified based on their ability to stimulate the proliferation of naive allogeneic T cells in a mixed leukocyte reaction (MLR).

In addition, is has been shown that, while immature dendritic cells are very efficient at antigen uptake but are poor antigen presenting cells, mature dendritic cells are poor at antigen uptake but are very efficient antigen presenting cells. Accordingly, the antigen presenting function of dendritic cells can be used to determine the degree of maturation. The antigen presenting function of a dendritic cell can be measured using antigen-dependent, MHC-restricted T cell activation assays as described herein, as well as other standard assays well known to those of skill in the art. T cell activation can further be determined, e.g., by measuring the induction of cytokine production by the stimulated dendritic cells. The stimulation of cytokine production can be quantitated using a variety of standard techniques, such as ELISA, well known to those of skill in the art.

F. General cell culture methods

The present invention relies on routine techniques in the field of cell culture, and suitable conditions can be easily determined by those of skill in the art (see, e.g., Freshney et al., Culture of Animal Cells, 3rd ed. (1994)). In general, the cell culture environment includes consideration of such factors as the substrate for cell growth, cell density and cell contract, the gas phase, the medium, the temperature, and the presence of growth factors.

Exemplary cell culture conditions for dendritic cells and dendritic cell precursors are described in, e.g., U.S. Pat. Nos. 6,017,527 and 5,851,756; Inaba et al., J. Exp. Med. 176:1693 (1992); Inaba et al., J. Exp. Med. 175:1157 (1992); Inaba et al., Current Protocols Immunol., Unit 3.7 (Coico et al., eds. 1998); Schneider et al., J. Immunol. Meth. 154:253 (1992); and Lutz et al., supra.

The cells of the invention can be grown under conditions that provide for cell to cell contact. In a preferred embodiment, the cells are grown in suspension as three dimensional aggregates. Suspension cultures can be achieved by using, e.g., a flask with a magnetic stirrer or a large surface area paddle, or on a plate that has been coated to prevent the cells from adhering to the bottom of the dish. For example, the cells may be grown in Costar dishes that have been coated with a hydrogel to prevent them from adhering to the bottom of the dish.

For cells that grow in a monolayer attached to a substrate, plastic dishes, flasks, roller bottles, or microcarriers are typically used. Other artificial substrates can be used such as glass and metals. The substrate is often treated by etching, or by coating with substances such as collagen, chondronectin, fibronectin, laminin or poly-L-lysine. The type of culture vessel depends on the culture conditions, e.g., multi-well plates, petri dishes, tissue culture tubes, flasks, roller bottles, microcarriers, and the like. Cells are grown at optimal densities that are determined empirically based on the cell type.

Important constituents of the gas phase are oxygen and carbon dioxide. Typically, atmospheric oxygen tensions are used for dendritic cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media, and is typically present at a concentration of 1-10% in the incubator. The preferred CO₂ concentration for dendritic cell cultures is 5%.

Cultured cells are normally grown in an incubator that provides a suitable temperature, e.g., the body temperature of the animal from which is the cells were obtained, accounting for regional variations in temperature. Generally, 37° C. is the preferred temperature for dendritic cell culture. Most incubators are humidified to approximately atmospheric conditions.

Defined cell media are available as packaged, premixed powders or presterilized solutions. Examples of commonly used media include Iscove's media, RPMI 1640, DMEM, and McCoy's Medium (see, e.g., GibcoBRL/Life Technologies Catalogue and Reference Guide; Sigma Catalogue). Defined cell culture media are often supplemented with 5-20% serum, e.g., human, horse, calf, or fetal bovine serum. The culture medium is usually buffered to maintain the cells at a pH preferably from about 7.2 to about 7.4. Other supplements to the media include, e.g., antibiotics, amino acids, sugars, and growth factors (see, e.g., Lutz et al., supra).

As described above, GM-CSF is typically added in concentrations ranging from 5 ng/ml to about 20 ng/ml. Other factors described herein and known to stimulate growth of dendritic cells may be included in the culture medium. Some factors will have different effects that are dependent upon the stage of differentiation of the cells, which can be monitored by testing for differentiation markers specific for the cell's stage in the differentiation pathway. GM-CSF is preferably present in the medium throughout culturing. Other factors that may be desirable to add to the culture medium include, but are not limited to, granulocyte colony-stimulating factor (G-CSF), M-CSF, TNF-α, IFN-γ, IL-1, IL-3, IL-6, SCF, LPS, and thrombopoietin. In some embodiments of the present invention, IL-4 is added to the culture medium, preferably at a concentration ranging from 1-100 ng/ml, most preferably from about 5 to about 20 ng/ml.

The present invention is also based in part on the surprising result that dendritic cell can be recovered and used after cryogenic storage. The present invention, thus, also provides methods for cryogenically storing precultured DCs, e.g., in liquid nitrogen, for several weeks. In a preferred embodiment, the dendritic cells are cultured in the presence of GM-CSF, preferably for 10 days, prior to being stored cryogenically. The DCs can be stored either as immature cells or, preferably, as matured APCs, following stimulation by suitable adjuvants, as described above. Furthermore, the DCs can be cryogenically stored either before or following exposure to an antigen of interest.

A variety of cryopreservation agents can be used and are described in, e.g., U.S. Pat. No. 5,788,963. Controlling the cooling rate, adding cryoprotective agents and/or limiting the heat of fusion phase where water turns to ice help preserve the function of the activated DCs. The cooling procedure can be carried out by use of, e.g., a programmable freezing device or a methanol bath procedure. After thorough freezing, cells can be rapidly transferred to a long-term cryogenic storage vessel. The samples can be cryogenically stored, for example, in liquid nitrogen (−196° C.) or its vapor (−165° C.). Such storage is greatly facilitated by the availability of highly efficient liquid nitrogen refrigerators. For a general description of methods to store DCs cryogenically see, e.g., U.S. Pat. No. 5,788,963.

IV. Antigen Stimulation

A. Pulsing the Antigen Presenting Cells With an Antigen of Interest

Following expansion in culture and maturation, the APCs of the present invention can further be pulsed with an antigen. APCs pulsed with an antigen of interest will process and present epitopes of the antigen. Antigens can be from any source, including, e.g., viruses, bacteria, parasites, etc. In one embodiment, the antigen is derived from Mycobacterium sp, Chlamydia sp., Leishmania sp., Trypanosoma sp., Plasmodium sp., or a Candida sp. APCs can be pulsed with either the entire peptide (antigen) or with a fragment thereof having immunogenic properties, e.g., an epitope.

Briefly, the antigen-activated APCs (e.g., antigen-activated dendritic cells) of the invention are produced by exposing, in vitro, an antigen to the APCs (e.g., the dendritic cells) prepared according to the methods of the invention. Dendritic cells, for example, are plated in culture dishes and exposed to an antigen of interest in a sufficient amount and for a sufficient period of time to allow the antigen to bind to the dendritic cells. The amount and time necessary to achieve binding of the antigen to the dendritic cells may be determined by using standard immunoassays or binding assays. Any other method known to those of skill in the art may also be used to detect the presence of antigen on the dendritic cells following their exposure to the antigen. Methods for pulsing dendritic cells with an antigen of interest are described, e.g., in U.S. Pat. No. 6,017,527.

B. Obtaining the Antigens

In general, antigens and fragments thereof may be prepared using any of a variety of procedures well known to those of skill in the art. For example, antigens can be naturally occurring and purified from a natural source.

Alternatively, antigens and fragments thereof can be produced recombinantly using a DNA sequence that encodes the antigen, which has been inserted into an appropriate expression vector, i.e., a vector which contains the necessary elements for the transcription and translation of the inserted coding sequence, and expressed in an appropriate host. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning: A Laboratory Manual, Cold Spring Harbor Laboratories, Cold Spring Harbor, N.Y. (1989); and Ausubel et al., Current Protocols in Molecular Biology (1995 supplement).

In addition, antigens and portions thereof may also be generated by synthetic means. Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain (see Merrifield, J. Am. Chem. Soc. 85:2149-2146 (1963)). Equipment for automated synthesis of polypeptides is commercially available from suppliers such as Perkin Elmer/Applied BioSystems Division, Inc., Foster City, Calif., and may be operated according to the manufacturer's instructions. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Within certain embodiments, the antigen of interest may be a fusion protein that comprises multiple polypeptides. A fusion protein may, for instance, include an antigen and a fusion partner which may, e.g., assist in providing T helper epitopes, and/or assist in expressing the protein at higher yields than the native recombinant protein. Other fusion partners may be selected so as to increase the solubility of the protein or to enable the protein to be targeted to desired intracellular compartments. Still further fusion partners include affinity tags, which facilitate the purification of the protein. Fusion proteins may generally be prepared using standard techniques, including chemical conjugation. Preferably, a fusion protein is expressed as a recombinant protein.

Furthermore, epitopes for use with the methods of the present invention can be selected based on the presence of specific MHC I and MHC II motifs well known to those of skill in the art.

C. Selecting the Antigens

In the context of the present invention, the antigens, antigen fragments or fusion proteins used to pulse the dendritic cells are preferably immunogenic, i.e., they are able to elicit an immune response (e.g., cellular or humoral) in a patient, such as a human, and/or in a biological sample (in vitro). In particular, antigens that are immunogenic (and portions of such antigens that are immunogenic) comprise an epitope recognized by a B-cell and/or a T-cell surface antigen receptor. Antigens that are immunogenic (and immunogenic portions of such antigens) are capable of stimulating cell proliferation, interleukin-12 production and/or interferon-γ production in biological samples comprising one or more cells selected from the group of T cells, NK cells, B cells and macrophages, where the cells have been previously stimulated with the antigen.

A variety of standard assays for measuring the immunogenic properties of a polypeptide of interest or of a portion thereof are available and known to those of skill in the art (see, e.g., Paul, Fundamental Immunology, 3d ed., Raven Press, pp. 243-247 (1993), and references cited therein).

V. Immune Responses Elicited By Dcs

In one aspect of the invention, the activated antigen presenting cells (e.g., the activated dendritic cells) are used to generate an immune response to an antigen of interest. An immune response to an antigen of interest can be detected by examining the presence, absence, or enhancement of specific activation of CD4+ or CD8+ T cells or by antibodies. Typically, T cells isolated from an immunized individual by routine techniques (e.g., by Ficoll/Hypaque density gradient centrifugation of peripheral blood lymphocytes) are incubated with an antigen. For example, T cells may be incubated in vitro for 2-9 days (typically 4 days) at 37° C. with the antigen. It may be desirable to incubate another aliquot of a T cell sample in the absence of the antigen to serve as a control.

Specific activation of CD4+ or CD8+ T cells may be detected in a variety of ways. Methods for detecting specific T cell activation include detecting the proliferation of T cells, the production of cytokines, or the generation of cytolytic activity (i.e., generation of cytotoxic T cells specific for an antigen). For CD4+ T cells, a preferred method for detecting specific T cell activation is the detection of the proliferation of T cells. For CD8+ T cells, a preferred method for detecting specific T cell activation is the detection of the generation of cytolytic activity.

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl)-2,5-diphenyltetrazolium. Alternatively, synthesis of lymphokines (e.g., interferon-gamma (IFN-γ)) can be measured or the relative number of T cells that can respond to the antigen may be quantified.

The secretion of IL-2 or IFN-γ can be measured by a variety of known techniques, including, but not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al., J. Biol. Chem. 255:4980-4983 (1980)); radioimmunoassays (RIA); enzyme-linked immunosorbent assays (ELISA) as described, for example, by Raines et al., J. Biol. Chem. 257:5154-5160 (1982); immunocytochemical techniques, including the use of fluorochromes (Brooks et al., Clin. Exp. Immunol. 39:477 (1980)); and neutralization of activity (Bowen-Pope et al., Proc. Natl. Acad. Sci. USA 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

VI. T Cells

The present invention is also directed to immunotherapeutic compositions comprising T cells specific for an antigen of interest. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also, U.S. Pat. Nos. 5,240,856 and 5,215,926; WO 89/06280; WO 91/16116; and WO 92/07243). Alternatively, T cells may be derived from related or unrelated humans, non-human mammals, cell lines or cultures.

T cells may be stimulated with a antigen of interest, a polynucleotide encoding an antigen of interest or, preferably, an antigen presenting cell (APC) that expresses such antigen. Such stimulation is performed under conditions and for a time sufficient to permit the generation of T cells that are specific for the antigen.

In a preferred embodiment of the invention, T cells are stimulated in vitro with isolated dendritic cells pulsed with an antigen of interest as described supra. The DCs can be used immediately after exposure to the antigen to stimulate T cells. Alternatively, the DCs can be maintained in the presence of a combination of GM-CS and IL-4 prior to simultaneous exposure to the antigen and the T cells. In preferred embodiments, the DCs are human DCs. The stimulated T cells can then be administered to a patient, for example, by intravenous infusion (see, Ridell et al., Science 257:238-241 (1992)). Infusion can be repeated at desired intervals such as, e.g., daily, weekly, monthly, etc. Recipients are monitored during and after T cell infusions for any evidence of adverse effects.

T cells are considered to be specific for an antigen if the T cells specifically proliferate, secrete cytokines or kill target cells coated with the antigen or expressing a gene encoding the antigen. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with an antigen or, preferably, with an activated DC for, e.g., 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience, Greene (1998)). T cells that have been activated in response to an antigen, polynucleotide or antigen-expressing APC may be CD4+ and/or CD8+. Antigen-specific T cells may be expanded using standard techniques. Within preferred embodiments, the T cells are derived from a patient, a related donor or an unrelated donor, and are administered to the patient following stimulation and expansion.

For therapeutic purposes, CD4+ or CD8+ T cells that proliferate in response to an antigen, polynucleotide or APC can be expanded in number either in vitro or in vivo. Proliferation of such T cells in vitro may be accomplished in a variety of ways. For example, the T cells can be re-exposed to an antigen, or a short peptide corresponding to an immunogenic portion of such an antigen, with or without the addition of T cell growth factors, such as interleukin-2, and/or stimulator cells that synthesize an antigen. Alternatively, one or more T cells that proliferate in the presence of an antigen can be expanded in number by cloning. Methods for cloning cells are well known in the art, and include, e.g., limiting dilution.

VII. Pharmaceutical Compositions

In one aspect of the invention, DCs are isolated from a patient, cultured and exposed in vitro to an antigen of interest, as described above, and after expansion and/or cryogenic storage are administered back to the patient to stimulate an immune response, including T cell activation, in vivo (see, e.g., Thurner et al., J. Immunol. Methods 223:1-15 (1999)).

The DCs obtained as described above are exposed ex vivo to an antigen, washed and administered to elicit an immune response or to augment an existing, albeit weak, response. As such, the DCs may constitute a vaccine and/or an immunotherapeutic agent. DCs presenting an antigen of interest can be administered using a variety of routes such as, for example, via intravenous infusion. The immune response of the patient can be monitored following DC administration. Infusion can be repeated at desired intervals based upon the patient's immune response. Methods for administering dendritic cells to a patient for eliciting an immune response in the patient are described, e.g., in U.S. Pat. Nos. 5,849,589; 5,851,756; 5,994,126; and 6,017,527.

In addition, antigen presenting cells (APCs) and in particular dendritic cells can be used as delivery vehicles for administering pharmaceutical compositions and vaccines. In this context, the APCs may, but need not, be genetically modified, e.g., to increase the capacity for presenting the antigen, to improve activation and/or maintenance of the T cell response and/or to be immunologically compatible with the receiver (i.e., matched HLA haplotype). APCs may generally be isolated from any of a variety of biological fluids and organs as described above, and may be autologous, allogeneic, syngeneic or xenogeneic cells.

APCs may generally be transfected with a polynucleotide encoding a antigen of interest (or portion thereof) such that the antigen, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460 (1997). Antigen loading of dendritic cells may be achieved by incubating dendritic cells or progenitor cells with the antigen, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### VI. EXAMPLES

### Example 1

### Human Dendritic Cell Studies

Recent articles have described how human dendritic cells can be derived in vitro from CD14 positive monocyte precursors in peripheral blood. A “buffy coat” preparation can now be commonly obtained from a unit of normal human peripheral blood from a blood bank on request. The leukocyte fraction was harvested from this buffy coat preparation using standard ficoll centrifugation techniques. Subsequently, the leukocyte fraction was labeled with anti-CD 14 antibody coupled to magnetic microbeads. This preparation was then passed over a magnet (Miltanyi Biotech) and CD14 positive monocytes were collected. These CD14 positive monocyte precursors were then cultured for 5-7 days in vitro (5×10⁵ cells/ml) with GM-CSF (20 ng/ml) for the duration of the culture. This type of in vitro expansion culture resulted in highly enriched dendritic cell outgrowth (5×10⁵/ml—65%-80% DCs). These immature DCs were then further stimulated in vitro for 24 hours in the presence of LPS (10 ng/ml), MPL® immunostimulant-S (100-200 ng/ml) or the ethanol-in-water formulation of RC-527, RC-529, and RC-544—all at 100 to 200 ng/ml. After the 24 hour in vitro stimulation culture, cells were washed twice and stained with a panel of monoclonal antibodies specific for different cell surface activation and co-receptor molecules found on mature, activated DCs.

Tables 1-3 below show data taken from three separate human DC expansion cultures (i.e., three separate normal donors) in which 7 day immature DCs were further cultured for 24 hours in the presence of LPS (10 ng/ml), MPL® immunostimulant-S (100 ng/ml), RC-527-S (100 ng/ml), RC-529-S (100 ng/ml) or RC-544-S (100 ng/ml) or were not treated with any additional stimulation (control culture). Flow cytometry analysis of control and stimulated normal monocyte-derived DCs indicated, in all three experiments, that there was a significant increase in the expression level of several important activation and co-stimulatory surface antigens as measured by increases in total number of positive cells or increase in mean channel fluorescence (MFI). Most notably, HLA-DR expression increases by 2-3 fold over control levels as measured by MFI changes. Three important co-stimulatory molecules, CD86, CD80 and CD40 also increased significantly compared to control cells in all three experiments—the co-stimulatory molecules were all required to mediate effective APC stimulation of antigen naive and (to a lesser degree) memory T cells. The substantial increase in the expression level of CD83, a hallmark indicator for DC maturation, after stimulation with LPS, MPL® immunostimulant or with the synthetic lipid A molecules, reveals the differentiation of immature DCs to more mature DCs capable of higher levels of antigenic presenting function.

In this series of experiments, the synthetic molecules RC-529 and RC-544 had very comparable effects, in most cases, in stimulating the expression of HLA-DR and co-stimulatory molecules. Overall RC-529 was slightly more effective at the upregulation of HLA-DR expression in all three experiments, while RC-544 stimulated slightly higher levels of CD40 and CD83 expression than did RC-529.

Table 4 shows correlated data in which 22 hour induction supernatants were collected from the same 7-day DC expansion cultures described in Tables 1-3. Supernatants from DCs stimulated with LPS, MPL® immunostimulant-S, RC-527, RC-529, and RC-544 were collected and tested by ELISA analysis for cytokines. Table 4 shows the results for IL-12 (p40), IL-12 (p70) and TNF-α analysis. In all three experiments, both LPS (10 ng/ml) and MPL® immunostimulant-S (100 ng/ml) stimulated comparable high levels of IL-12 (p40) expression above background. RC-527 (100 ng/ml) was in every case a potent stimulator of IL-12 (p40), and RC-529 and RC-S44 also stimulated IL-12 (p40) responses.

The IL-12(p70) response in these induction cultures was more complex. Control levels were essentially zero, but LPS and MPL® immunostimulant-S stimulated a quantitatively significant amount of IL-12 (p70) in only one donor. In that experiment RC-527, RC-529 and RC-544 all stimulated high levels of IL-12 (p70). In the other two experiments (using different normal donors), RC-527 stimulated significant levels of IL-12 (p70) expression. Table 4 also shows that both LPS and MPL® immunostimulant-S induced significant TNF-α secretion. RC-527 stimulated a very strong TNF-α effect in all three donors. The TNF-α effect due to RC-529 and RC-544 was comparable in one experiment and moderately lower than the effect due to RC-527. In a second experiment the RC-529 and RC-544 TNF-α induction effect was significant.

TABLE 1
Human d7 Cultured Dendritic Cells (092398) Flow Cytometry Phenotype Analysis

| | | No Treatment | LPS | MPL ® | 527 | 529 | 544 |
|---|---|---|---|---|---|---|---|
| HLA-DR | MFI | 53.9 | 147.8 | 155.9 | 107.8 | 158.8 | 155.2 |
| CD4 | % | 86.4% | 70.6% | 69.4% | 69.5% | 71.6% | 75.7% |
| | MFI | 5.20 | 2.58 | 2.58 | 2.66 | 2.65 | 2.80 |
| CD 86 | % | 79.1% | 99.9% | 99.8% | 99.9% | 99.9% | 99.9% |
| | MFI | 5.07 | 21.2 | 21.6 | 28.0 | 25.3 | 26.4 |
| CD 80 | % | 56.2% | 99.1% | 99.0% | 99.4% | 99.1% | 99.1% |
| | MFI | 2.27 | 8.93 | 8.53 | 12.7 | 8.46 | 7.56 |
| CD 14 | % | 34.4% | 5.6% | 4.2% | 4.3% | 4.2% | 5.0% |
| CD 40 | MFI | 6.16 | 23.5 | 25.8 | 35.0 | 31.5 | 33.7 |
| CD 83 | % | 22.0% | 87.8% | 90.5% | 97.6% | 94.7% | 97.1% |
| | MFI | 1.94 | 3.55 | 3.52 | 3.57 | 3.32 | 3.52 |
| CD 62 L | % | 5.8% | 10.9% | 8.9% | 13.6% | 11.1% | 7.1% |
| CD 1a | % | 83.5% | 88.1% | 87.7% | 84.1% | 86.5% | 75.6% |
| | MFI | 6.38 | 6.36 | 5.90 | 5.57 | 5.47 | 4.44 |
| CD 69 | % | 5.6% | 7.4% | 6.9% | 10.5% | 11.0% | 10.1% |
| CD 95 | % | 47.3% | 83.9% | 88.5% | 94.8% | 90.4% | 90.6% |
| | MFI | 1.21 | 1.37 | 1.39 | 1.78 | 1.43 | 1.40 |
| CD 44 | MFI | 296+ | 497+ | 485+ | 499+ | 442+ | 426+ |
| CD 35 | % | 64.3% | 60.3% | 60.5% | 65.1% | 58.8% | 59.1% |
| | MFI | 3.68 | 2.92 | 2.93 | 3.02 | 2.92 | 2.98 |
| CD 54 | MFI | 18.0 | 56.9 | 51.6 | 42.6 | 36.6 | 32.7 |
| GAR-Alexa | | 39% | 3.6% | 2.0% | 2.9% | 2.4% | 3.9% |
| αC5aR + GAR | | 73.9% | 52.7% | 51.6% | 42.3% | 53.4% | 45.9% |
| Alexa | | 9.49 | 9.58 | 12.7 | 8.44 | 10.5 | 10.8 |

*Mean channel fluorescence values only are given for HLA-DR, CD 40, and CD 44 since >95% of all cells are positive for the indicated markers.

TABLE 2
Human d7 Cultured Dendritic Cells (091698) Flow Cytometry Phenotype Analysis

| | | No Treatment | LPS | MPL ®-S | 527-S | 529-S | |
|---|---|---|---|---|---|---|---|
| HLA-DR | MFI | 48.4 | 86 | 69.4 | 74.2 | 88.2 | 544-S 60.8 |
| CD 4 | % | 86.0% | 70.8% | 85.6% | 69.5% | 77.0% | 70.6% |
| | MFI | 4.21 | 2.48 | 2.89 | 2.33 | 2.44 | 2.17 |
| CD 86 | % | 69% | 98.4% | 94.6% | 99.8% | 98.7% | 99.7 |
| | MFI | 3.87 | 9.13 | 7.43 | 18.1 | 9.30 | 15.3 |
| CD 80 | % | 83.7% | 99.0% | 97.2% | 99.5% | 98.9% | 99.2% |
| | MFI | 3.15 | 7.93 | 5.22 | 8.37 | 6.19 | 5.99 |
| CD 14 | % | 18.1% | 10.9% | 3.2% | 4.2% | 7.5% | 7.6% |
| CD 40 | MFI | 6.98 | 20.5 | 15.2 | 43.0 | 22.0 | 31.8 |
| CD 83 | % | 14.7% | 67.9% | 60.2% | 97.0% | 71.0% | 94.3% |
| | MFI | 1.82 | 3.21 | 2.68 | 3.52 | 2.89 | 2.72 |
| CD 62 L | % | 4.9% | 6.9% | 6.5% | 11.2% | 6.4% | 11.6% |
| CD 1a | % | 95.1% | 94.3% | 93.1% | 93.5% | 94.3% | 11.6% |
| | MFI | 11.8 | 11.7 | 9.58 | 8.71 | 9.29 | 7.38 |
| CD 69 | % | 3.7% | 6.7% | 3.2% | 16.1% | 4.4% | 10.8% |
| CD 95 | % | 31.4% | 65.8% | 42.5% | 93.6% | 53.8% | 86.0% |
| | MFI | 1.12 | 1.17 | 1.10 | 1.61 | 1.12 | 1.31 |
| CD 44 | MFI | 160 | 286 | 242 | 329 | 285 | 242 |
| CD 35 | % | 55.6% | 46.9% | 46.7% | 49.4% | 47.6% | 52.5% |
| | MFI | 3.06 | 2.72 | 2.78 | 2.74 | 2.66 | 2.85 |
| CD 54 | MFI | 12.8 | 27.8 | 23.6 | 34.9 | 28.2 | 26.1 |

*Mean channel fluorescence values only are given for HLA-DR, CD 40, and CD 44 since >95% of all cells are positive for the indicated markers.

TABLE 3
Human d7 Cultured Dendritic Cells (093098) Flow Cytometry Phenotype Analysis
Day 7 RC-093098

| | | No Treatment | LPS | MPL ®-S | 527 | 529 | 544 |
|---|---|---|---|---|---|---|---|
| HLA-DR | MFI | 66.2 | 171.2 | 160.1 | 172.9 | 161.5 | 134.3 |
| CD 4 | % | 88.4% | 82.5% | 84.0% | 75.2% | 77.9% | 79.4% |
| | MFI | 5.14 | 3.28 | 3.46 | 3.06 | 2.97 | 3.11 |
| CD 86 | % | 73.5% | 99.0% | 97.9% | 99.5% | 99.7% | 99.8% |
| | MFI | 8.16 | 17.2 | 16.3 | 27.4 | 22.0 | 26.3 |
| CD 80 | % | 85.1% | 98.7% | 98.2% | 98.9% | 99.1% | 99.0% |
| | MFI | 4.68 | 10.6 | 8.75 | 13.1 | 10.7 | 10.9 |
| CD 14 | % | 19.9% | 12.7% | 7.5% | 3.3% | 3.9% | 4.1% |
| CD 40 | MFI | 7.99 | 20.4 | 18.1 | 34.9 | 26.1 | 30.4 |
| CD 83 | % | 28.0% | 69.1% | 65.5% | 92.6% | 82.4% | 92.5% |
| | MFI | 2.47 | 3.59 | 3.47 | 3.63 | 3.38 | 3.43 |
| CD 62L | % | 5.4% | 4.0% | 4.7% | 3.0% | 3.4% | 4.0% |
| CD 1a | % | 82.1% | 82.6% | 83.1% | 80.3% | 80.1% | 78.1% |
| | MFI | 7.65 | 7.55 | 7.83 | 6.83 | 6.51 | 6.06 |
| CD 3 | % | 40.2% | 28.2% | 29.0% | 28.5% | 27.3% | 26.8% |
| | MFI | 4.54 | 3.96 | 3.98 | 3.89 | 3.87 | 3.86 |
| CD54 | MFI | 15.3 | 41.5 | 36.7 | 47.0 | 36.4 | 36.8 |
| CD 11c | % | 82.7% | 92.7% | 91.1% | 92.6% | 92.4% | 91.0% |
| | MFI | 8.83 | 7.11 | 7.11 | 6.77 | 6.82 | 6.23 |
| CD 123 | % | 18.0% | 29.8% | 29.6% | 52.9% | 44.1% | 48.0% |
| | MFI | 1.86 | 1.80 | 1.83 | 1.99 | 1.90 | 1.85 |
| CCR 6 | % | 4.3% | 4.8% | 5.7% | 4.0% | 5.3% | 5.8% |
| αC5aR + Alexa | % | 48.1% | 49.5% | 38.2% | 42.7% | 48.6% | 43.3% |
| 488 | MFI | 19.3 | 9.82 | 11.9 | 8.17 | 10.4 | 9.41 |

*Mean channel fluorescence values only are given for HLA-DR, CD 40, and CD 44 since >95% of all cells are positive for the indicated markers.

TABLE 4

| | | | | | | |
|---|---|---|---|---|---|---|
| Exp. # | No Treatment | LPS | MPL ®-S | 527 | 529 | 544 |

IL-12 p40 pg/mL/5 × 10⁵ Cells - 22 Hrs

| | | | | | | | |
|---|---|---|---|---|---|---|---|
| 091698 | | 217 | 35,600 | 14,700 | 202,200 | 38,700 | 42,500 |
| 092398 | Day 7 | 112 | 68,600 | 68,300 | 163,000 | 127,500 | 80,500 |
| 093098 | | 216 | 29,200 | 12,800 | 107,600 | 60,700 | 62,000 |

IL-12 p70 pg/ml/5 × 10⁵ Cells - 22 Hrs

| | | | | | | | |
|---|---|---|---|---|---|---|---|
| 092398 | | ND | 1.9 | ND | 194.0 | 4.2 | 8.8 |
| 092398 | Day7 | ND | 127.7 | 104.9 | 201.4 | 215.3 | 175.2 |
| 092398 | | ND | 1.2 | 1.4 | 168.9 | 28.2 | 55.7 |

TNF-α pg/mL/5 × 10⁵ Cells - 22 Hrs

| | | | | | | | |
|---|---|---|---|---|---|---|---|
| 093098 | | ND | 930 | 112 | 4,000 | 372 | 880 |
| 092398 | Day 7 | ND | 3,940 | 2,600 | 10,750 | 7,400 | 5.650 |
| 093098 | | ND | 2,140 | 380 | 7,020 | — | — |

ND = not detectable. Data show comparative effects of the indicated compounds to stimulate IL-12 (p40),
IL-12 (p70) and TNF-α from 6-7 day cultured human dendritic cells. Supernatants were collected after 22 hrs of induction.

### Example 2

### Murine Dendritic Cell Studies

Using well established procedures, protocols were developed to grow large numbers of murine dendritic cells (DCs) from bone marrow derived precursor cells. Bone marrow was harvested aseptically from the tibias of female Balb/c mice (4-12 weeks) and cultured under standard in vitro culture conditions at 5×10⁵ cells/ml in T75 tissue culture flasks. Murine GM-CSF (20 ng/ml) and IL-4 (20 ng/ml) was added to the cultures to promote the outgrowth of DCs over a 6-7 day culture period. As with the human DC cultures, these cells developed into immature DCs at the end of 6-7 days in culture. These cells were, thus, lineage negative (i.e., non-T, non-B, non-NK) by standard phenotype specific cell surface staining criteria. They displayed the typical DC cell surface phenotype of CD11b⁺ and CD11⁺, and also showed high cell surface expression of Class II MHC antigen (I-A^{d}), and the co-stimulatory molecules CD80, CD86, and CD40. It has been shown that DCs cultured by this technique can be further triggered by LPS stimulation to significantly upregulate the cell surface expression for I-A^{d}, CD80, CD86 and CD40. In these experiments, however, neither LPS (R595) or MPL® immunostimulant-S stimulated any significant upregulation of the cell surface expression of relevant activation or co-stimulatory molecules on these murine bone marrow derived DCs. By 6-7 days in culture, those cells were already stimulated to display a relatively mature phenotype.

Even though LPS or MPL® (immunostimulant in vitro stimulation of the murine DCs did not appear to further substantially affect the maturation of these cells as assessed by change in cell surface phenotype, this type of in vitro potentiation with lipid A-like molecules did substantially increase their functional activity as antigen presenting cells. Several experiments were carried out in which 6-7 day bone marrow derived DCs were subsequently pulsed in vitro with ova (50 μg/ml-4 hours) and then cultured overnight in the presence of LPS (10 ng/ml) MPL® immunostimulant-S (100-200 ng/ml) or RC-527, RC-529, and RC-544 (all at 100-200 ng/ml). These antigen pulsed, lipid A potentiated cells were then washed thoroughly and titered against ova-specific T cell effector cells in vitro at an APC: T cell ratio titration of 1:1 out to as high as 1:512. The effector T cells in this in vitro assay were the antigen naive, but antigen (ova) specific, MHC restricted I-A^{d} splenic T cells from transgenic D011.10 mice.

D011.10 TCR-transgenic mice were used in these experiments as donors of antigen specific naive T cells. In their germline DNA these mice contain rearranged TCR-Vα and -Vβ genes that encode a T cell receptor (TCR) specific for the ovalbumin peptide 323-339 bound to IA^{d} (Balb/c) Class II MHC molecules. The transgenic ova peptide specific TCR can be detected with the KJ1-26 monoclonal antibody (mAb) that binds only to this particular TCR heterodimer. These cells provide a ready source of “normal” antigen (ova)-specific effector T cells. Effective antigen-mediated APC stimulation of those T cells is generally measured by ELISA quantitation of IL-2 or IFN-γ in the culture supernatants of DC+D011.10 T cells after 24-48 hours of incubation.

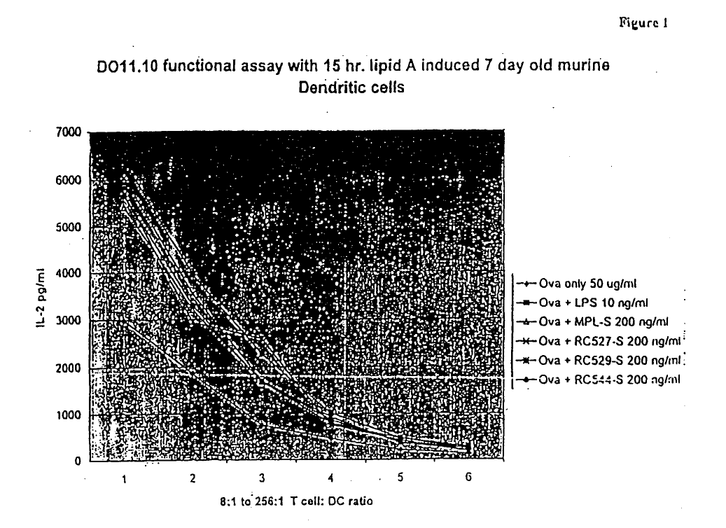

In one experiment, murine bone marrow derived DCs (6 day cultures) were pulsed with ova (50 μg/ml-5×10⁵ cells/ml) and then potentiated overnight with LPS(R595), MPL® immunostimulant-S, RC-527, RC-529 or RC-544 prior to being cultured together with naive D011.10 effector T cells (FIGS. 1-7). The APC function of these DCs as measured by their ability to stimulate IL-2, IFN-γ IL-12 (p70) and IL-12 (p40) secretion in the DC+D011.10 co-culture systems was analyzed. All of the data shown in FIGS. 1-7 was developed from DCs maintained in lipid A induction cultures for either 15 or 38 hours prior to use in the T cell assay.

Culture supernatants of DCs+D011.10 T cells were analyzed by an IL-2 ELISA (FIG. 1). DCs were maintained for 15 hours prior to the T cell assay in the presence of different induction stimuli. LPS generally stimulated high overall IL-2 secretion effect at all T cell:APC ratios tested, and this effect was higher than the response stimulated by DCs prestimulated with ova only (FIG. 1). All the other DC prestimulation test groups (i.e., MPL® immunostimulant-S, RC-527, RC-529, and RC-544) were comparable in their ability to stimulate IL-2 secretion at all T cell:APC ratios.

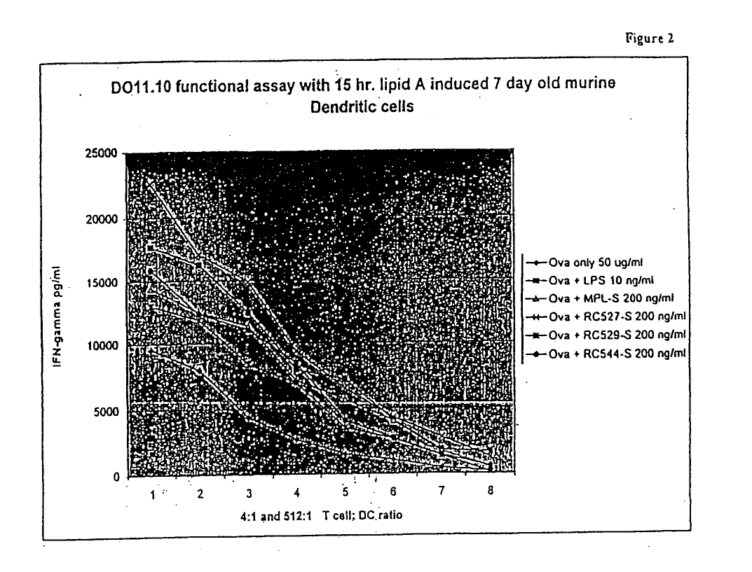

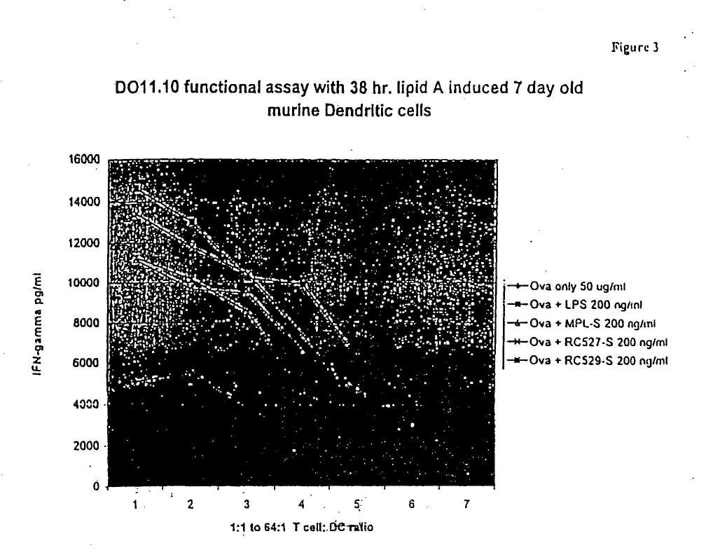

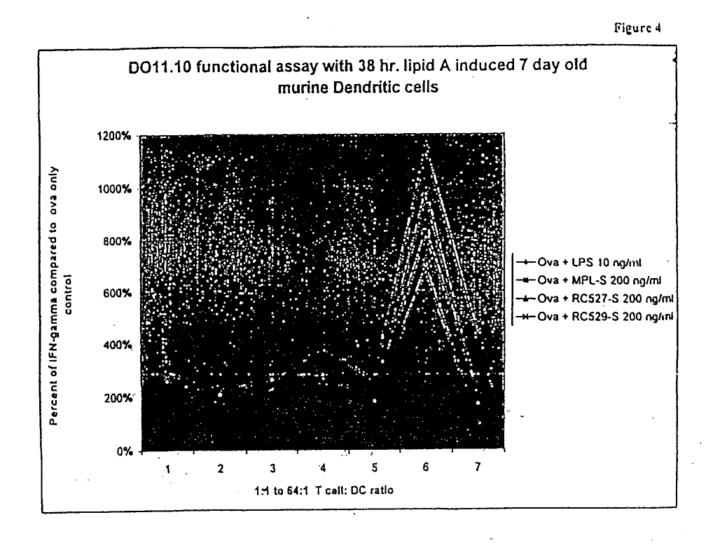

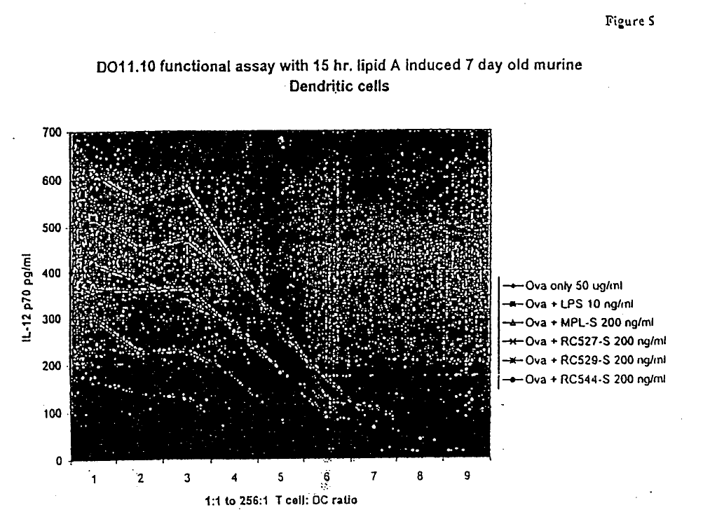

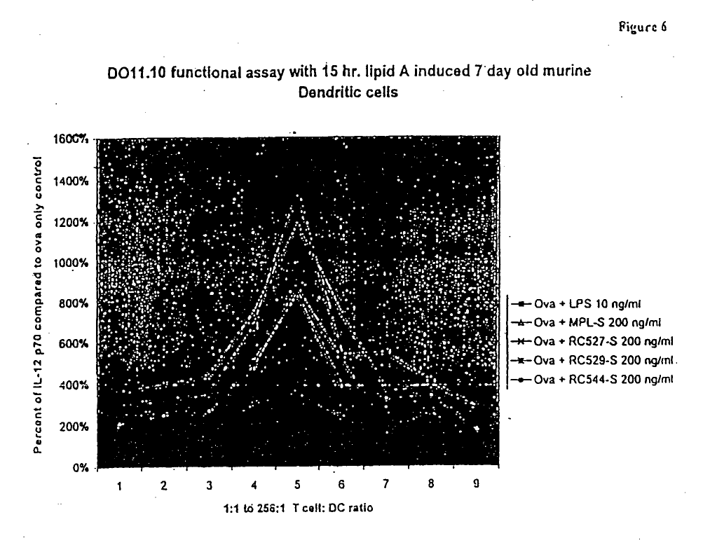

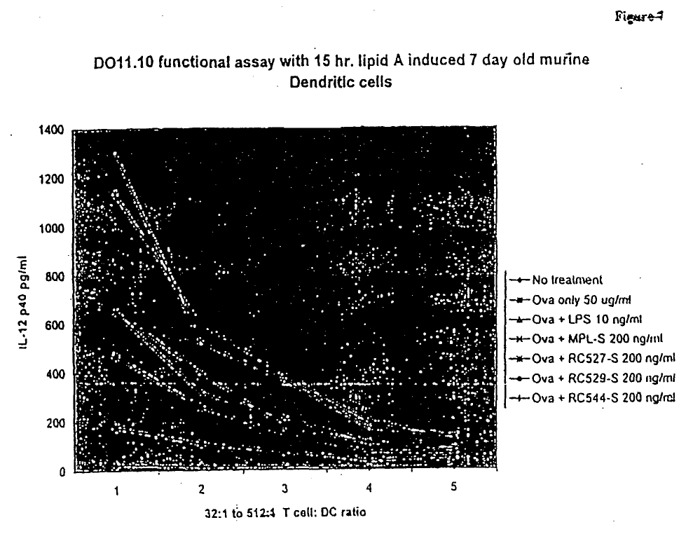

The effect of ova pulsed, 15 hour lipid A prestimulated DCs on IFN-γ secretion by D011.10 cells was then determined. RC-527 and LPS precultured DCs stimulated the high levels of IFN-γ release by D011.10 T cells, and this effect was higher than that observed for ova only treated DCs (FIG. 2). At 15 hours of preculture the DCs stimulated by all the other lipid A molecules tested were very comparable (at all T cell:APC ratios) in their ability to stimulate IFN-γ secretion (FIG. 2). Similarly DCs precultured for 38 hours in the presence of lipid A molecules also showed a comparable effect to the 15 hour DC stimulation results in driving IFN-γ release by D011.10 T cells (FIG. 3). Again, LPS and RC-527 were generally more potent in stimulating DC mediated IFN-γ secretion by the effector T cells than MPL® immunostimulant-S and RC-529, which were very comparable to each other. All DC stimulation regimens produced DCs with significantly higher APC function than that observed for DCs pulsed with ova only. Further analysis showed that the most effective T cell:APC ratio for the stimulation of IFNγ secretion was 32:1 (FIG. 4). At this ratio the effect of LPS-stimulated, ova-pulsed DCs was 10-12 fold greater than the effect of DCs pulsed with ova only. As shown, the effect of DC pre-stimulation with RC-527 was comparable to that observed with LPS at this T cell:APC ratio, and MPL® immunostimulant-S and RC-529 preconditioning of DCs induced comparable stimulation of IFN-γ secretion (i.e., 7-8 fold increase over ova pulsed DCs).

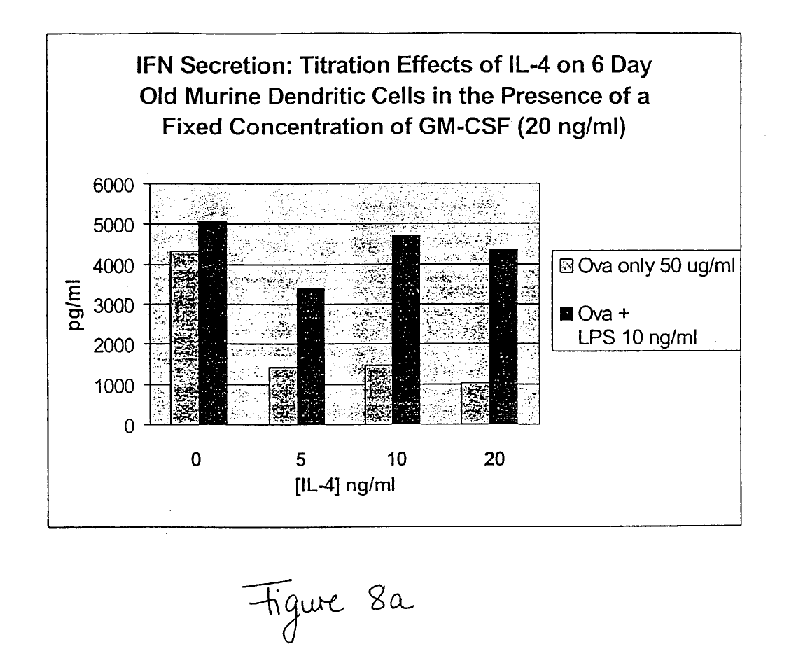

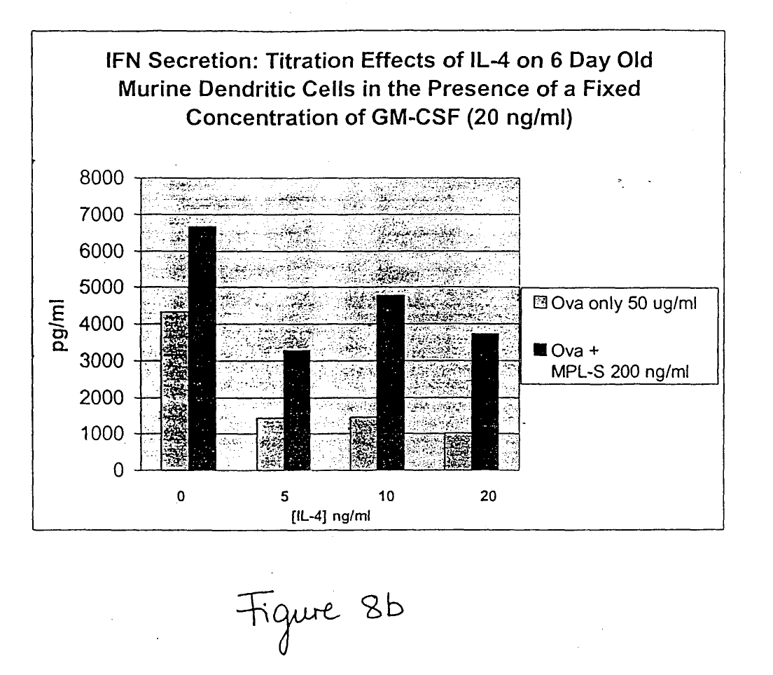

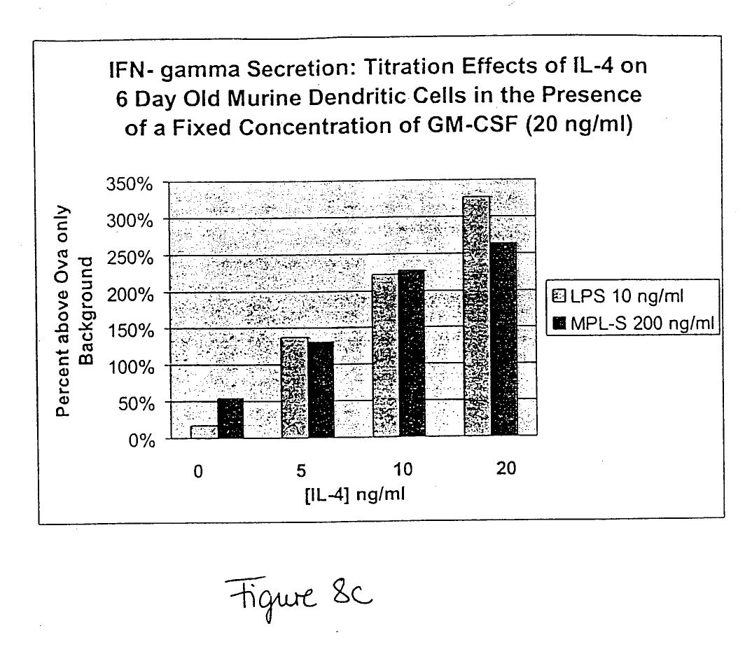

Additional experiments showed that lipid A precultured DCs in some manner produced the appearance of relatively high levels of the functionally active IL-12 (p70) when co-cultured with D011.10 T lymphocytes. This result was unexpected since repeated earlier efforts to measure secreted IL-12 (p70) in the supernatants of DCs directly stimulated with LPS or MPL® immunostimulant-S were unsuccessful. Analysis of “direct stimulation” supernatants showed the secretion of high levels of IL-12 (p40) homodimer, but not of the functionally active form of the molecule. Although T cells are generally not thought to be a significant source of IL-12, recent data has shown that once DC-T cell contact occurs, the DC can in fact secrete IL-12 (p70), probably via signal transduction occurring through CD40 (DC)-CD40 ligand (T cell) interaction. LPS and RC-527 precultured DCs were the most potent for inducing IL-12 secretion (FIG. 5). MPL® immunostimulant-S and RC-544 were very comparable, and overall weaker in their effect than LPS and RC-527. RC-529 showed the weakest ability to stimulate the DC mediated release of IL-12 (p70) in the DC+D011.10 cultures. All lipid A molecules were more effective in stimulating DC competence for this response than ovalbumin alone. The most effective T cell:APC ratio for this cytokine response was shown to be 16:1 (FIG. 6). Thus, RC-527 and LPS induce the DC-mediated IL-12 (p70) secretion response 12-14 times greater than the effect observed for ova only treated DCs. All the other lipid A molecules were most effective at this T cell:APC ratio with MPL® immunostimulant-S and RC-544, showing a substantially higher stimulation effect than that induced by RC-529.

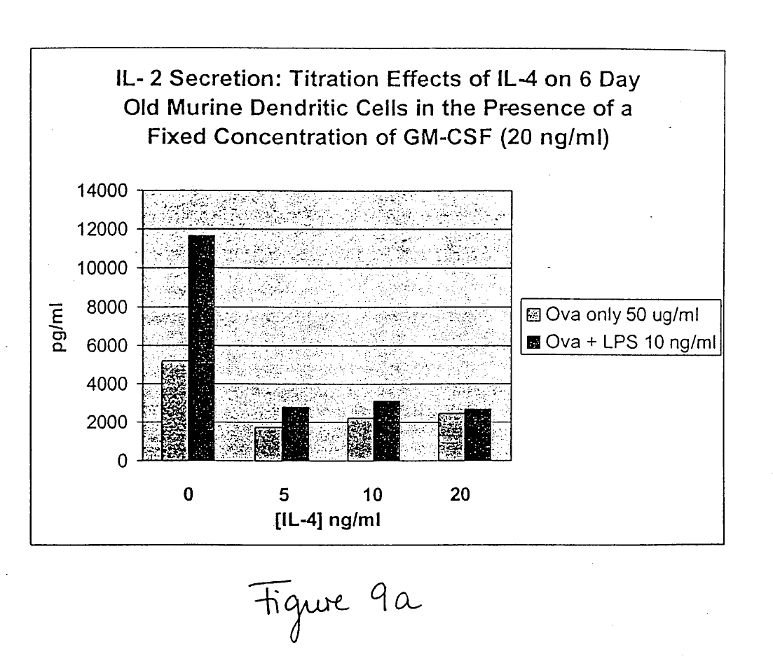

IL-12 (p40) was also shown to be released in the DC+D011.10 co-culture systems. LPS and RC-527 again had the most potent effect in stimulating the release of IL-12 (p40) homodimer (FIG. 7). MPL® immunostimulant-S and RC-544 were comparable to each other, but weaker in their effect than LPS and RC-527. PC-529 was also moderately less potent than either RC-544 or MPL® immunostimulant-S. All lipid A molecules stimulated greater IL-12 (p40) responses than those observed for DCs precultured with antigen only.

### Example 3

### D011.10 Into Balb/c-In Vivo Transgenic T Cell Transfer Experiments

An ova peptide-specific transgenic T cell receptor (TCR) murine D011.10 T lymphocyte model has been developed and can now be used routinely to study broad questions of T lymphocyte-APC interaction, as well as the role of different adjuvants and adjuvant formulations on the regulation of APC function. In particular, the model is useful to assess the effects of different adjuvant materials in regulating Th1 versus Th2 helper T cell response, and the effect of adjuvants in the strength of long-term, antigen-specific anamnestic response.

Initial experiments demonstrated the ability to successfully transfer the ova-specific TCR positive CD4 T lymphocytes from a transgenic D011.10 mouse into a syngeneic Balb/c mouse (I-A^{d}), and subsequently detect ova specific T cell amplification after immunizing the host Balb/c mouse with ova plus a potent adjuvant. In the first experiment Balb/c mice received different concentrations of D011.10 positive, CD4 lymphocytes by IV injection (Table 5). Twenty four hours later recipient Balb/c mice were immunized subcutaneously by bilateral inguinal injection with ova plus Detox adjuvant. Three and five days later inguinal lymph nodes were collected, cell counts per node were made and pooled lymph node cells were stained for CD4 (FITC) and KJ1 (APC) simultaneously. The expansion of KJ1*CD4 T lymphocytes was optimized by day 5, compared to day 3 (Table 5). Furthermore, there was an obvious dose response, since increasing numbers of injected D011.10 T cells KJ1-positive result in an increased expression of KJ1 positive T cells in the lymph node over time (Table 5).

In a second experiment 6-7 day bone marrow derived (Balb/c) DCs were pulsed with antigen and subsequently stimulated overnight with LPS, MPL® immunostimulant-S, and RC-527 (Table 6). 5×10⁶ D011.10 CD4 (KJ1-positive) T cells were transferred into Balb/c mice, and 24 hours later ova-pulsed cultured, DCs from the control group (pulsed with ova only) and the indicated test DCs potentiated with LPS, MPL® immunostimulant-S or RC-527 were also injected intravenously. Spleen cells were examined 3 and 5 days later by flow cytometry and the number of KJ 1-positive CD4 cells per spleen was enumerated. ova only, ova+LPS and ova+MPL® immunostimulant-S had comparable effects at day 3 in stimulating transferred DCs. The transferred DCs stimulated with ova only, ova+LPS and ova+MPL® immunostimulant-S produced a similar effect in inducing the amplification of D011.10 T lymphocyte numbers above the value observed when untreated DCs (i.e., no ova and no lipid A) were used as the putative APC source and transfected into Balb/c mice preinjected with KJ1 CD4 cells. By day 5 after the transfer of stimulated DCs, the ova only control DCs lost the ability to sustain KJ1⁺ CD4 cell numbers, whereas DCs treated with MPL® immunostimulant-S or LPS demonstrated a longer duration effect to maintain KJ1 cell numbers. In contrast, RC-527-stimulated DCs produced higher levels of splenic KJ1⁺ CD4 T cell numbers than any of the other test groups at both time points.

In a second, similar DC transfer experiment, comparable results were obtained, with ova only pulsed control DCs producing a KJ1⁺ CD4 response of 1.5% of all splenic CD4 cells. LPS treated (10 ng/ml) and MPL® immunostimulant-S treated (100 ng/ml) DCs each sustained a 3.0% KJ1⁺ CD4 response at 5 days, and RC-527 stimulated DCs produced a KJ1⁺ response of 3.9% of all splenic CD4 T cells at 5 days. Flow cytometry analysis showed the high degree of KJ1 specific fluorescence and allowed for a more clear-cut understanding of the ease with which D0-11.10 (KJ1 positive) CD4 T cells can be resolved from the total CD4 lymphocyte background.

TABLE 5
Titration of D011.10 Spleen Cells Into Balb/c Mice
Immunized With Detox ™ + OVA

% KJ1 Positive
CD4 Cells Per LN

| # of D011.10 Cells Transferred | Day 3 | Day 5 |
|---|---|---|
| 2.5 × 10⁶/mouse | 2.8% | 8.00% |
| 5.0 × 10⁶/mouse | 6.7% | 13.6% |
| 10.0 × 10⁶/mouse | 9.7% | 15.3% |

Balb/c mice were injected with D011.10 spleen cells as indicated above on day zero minus 1; 24 hrs later mice were injected with 200 μl/mouse (subcutaneously) of DetoX™ (neat)+OVA at 250 μg/ml. Inguinal lymph nodes were harvested bilaterally 3 and 5 days later and analyzed for % KJ1 positive CD4 cells.

TABLE 6
OVA Pulsed DC Transfer into D011.10 Loaded Balb/c Mice

Total KJ1 Positive
Cells/Spleen

| DC Treatment Test Groups | Day 3 | Day 5 |
|---|---|---|
| No OVA Treatment | 0.59 × 10⁶ | 0.68 × 10⁶ |
| OVA Only (SO μg/ml/10⁶ DCs) | 2.4 × 10⁶ | 1.2 × 10⁶ |
| OVA + LPS (10 ng/ml) | 2.5 × 10⁶ | 2.3 × 10⁶ |
| OVA + MPL ®-S (100 ng/ml) | 2.3 × 10⁶ | 2.4 × 10⁶ |
| OVA + RC-527 (100 ng/ml) | 4.1 × 10⁶ | 3.0 × 10⁶ |

5×10⁶ KJ1 positive D011.10 spleen cells were injected into Balb/c mice (I.V.) on day zero minus 1; 24 hrs later 5×10⁶ DC's from the indicated in vitro induction test groups were injected I.V. and the spleens from test animals were harvested 3 and 5 days later for total KJ1 enumeration.

### Example 4

### Murine Bone Marrow Derived Dendritic Cell Studies

It has been recently suggested that the treatment of murine bone marrow with GM-CSF alone causes outgrowth of “mixed” macrophage and immature DC cultures, while the addition of IL-4 suppresses macrophage growth (and probably macrophage function) and promotes the outgrowth of more mature DCs. In this context, a series of experiments were performed to test the stimulatory effects of LPS and MPL® immunostimulant on 6 day DC cultures grown in GM-CSF only or in GM-CSF and increasing concentrations of IL-4 (i.e., 5-20 ng/ml). Standard 6 day DC cultures were thus set up in GM-CSF alone at 20 ng/ml or with GM-CSF and 5, 10 or 20 ng/ml of IL-4. At the end of the 6 days, the nonadherent DCs were collected, pulsed with ova for 4 hours and (in the same culture dish) treated with either LPS (10 ng/ml) or MPL® immunostimulant-S (100 ng/ml). These DCs were then collected, washed and placed against ova-specific D011.10 splenic T cells at ratios of 4:1 to 128:1 (T cells:DC) in a 96 well format. Culture supernatants were collected from the DC-T cell 96 well cultures after 24 hours and tested by ELISA for INF-γ, IL-2 and IL-12 p70.

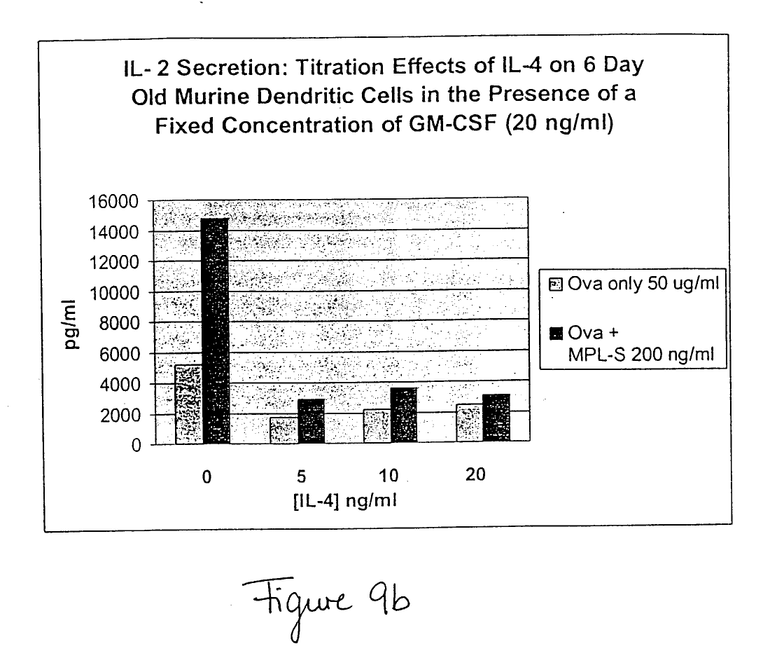

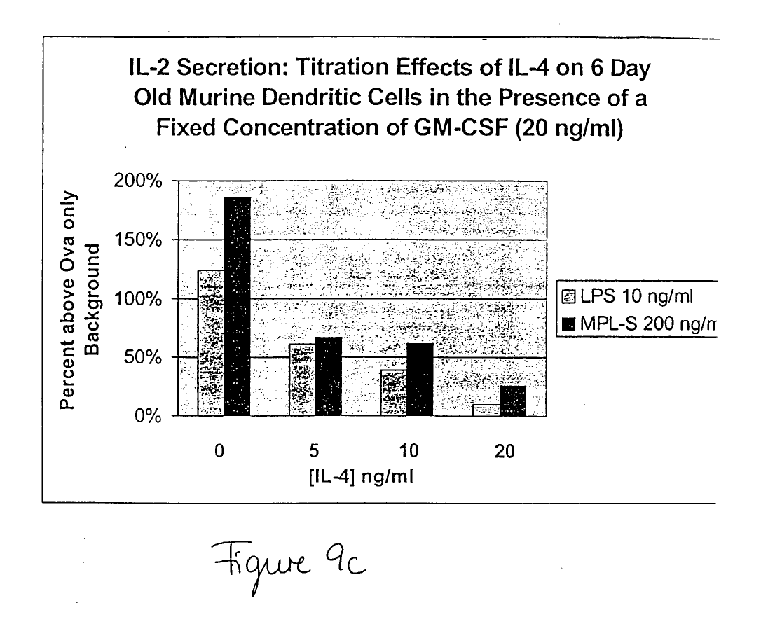

All the DC test groups (i.e., GM-CSF alone, and GM-CSF+IL-4 at 5, 10 or 20 ng/ml) were tested in a single experiment at a 8:1 ratio of D011.10 effector T cells to DCs. LPS- and MPL® immunostimulant-activated DCs stimulated relatively comparable absolute levels of IFN-γ secretion (i.e., between about 3,500 and 6,500 pg/ml) for all DC test groups (FIG. 8). These experiments showed that DCs cultured in GM-CSF alone displayed a much higher ova-only control background effect compared to the relatively uniform but much lower ova-only control effect for DCs grown at all concentrations of IL-4. LPS- and MPL® immunostimulant-treated DCs (8:1 T cell to DC ratio) were shown to have IFN-γ-inductive effects on all DC test populations relative to their respective ova-only DC background controls (FIG. 8c). The data is presented as the % of IFN-γ secretory response triggered by LPS- or MPL® immunostimulant-treated DCs above the IFN-γ response stimulated by ova antigen-only pulsed control DCs harvested from each of the four different cytokine expansion cultures. A stepwise increase in LPS- and MPL® immunostimulant-induced DC-mediated APC function (as measured by IFN-γ secretion) above the APC function of DCs pulsed with antigen-only was observed. This stepwise increase of the DC-mediated APC function correlated with the increasing dose of IL-4 used to maintain the initial expansion cultures of DCs. The LPS-induced absolute IFN-γ effect for GM-CSF only DCs was comparable to that measured for DCs grown in GM-CSF plus the two highest doses of IL-4. DCs cultured in GM-CSF plus IL-4 at 20 ng/ml produced a significantly lower absolute level of IFN-γ than DCs grown in GM-CSF only (FIG. 8b). However, because the ova-only control DC stimulated IFN-γ response was much lower in the DC test groups grown in IL-4, the overall comparative result was that both LPS and MPL® immunostimulant had the most potent stimulation effect (compared to ova-only controls) on more “mature” DCs grown in IL-4.

### Example 5

### LPS and MPL® Stimulation of IL-2 Secretion Mediated by 6 Day Murine Bone Marrow Derived DCs

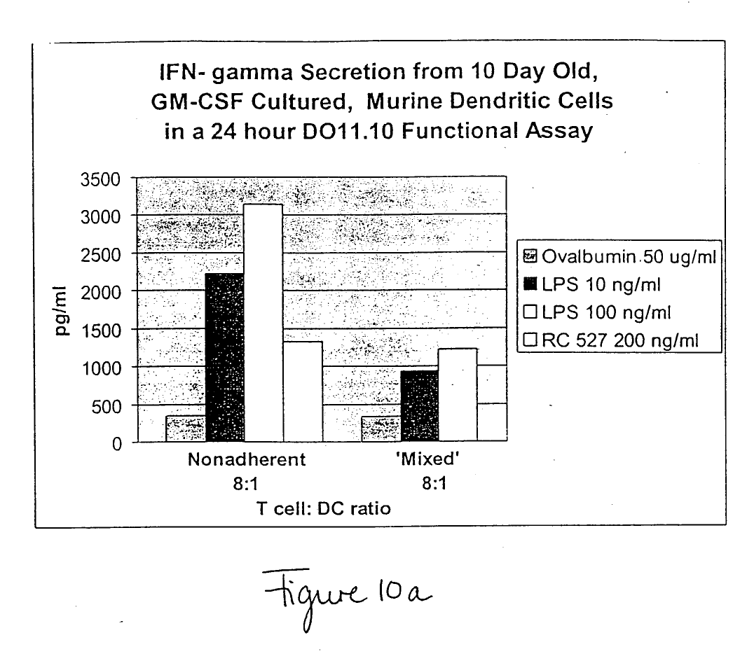

The same culture supernatants used to quantitate IFN-γ by ELISA in the experiments described above were also used to measure IL-2 cytokine levels, also by ELISA (FIGS. 9a-c). D011.10 T cells were stimulated for 24 hours in the presence of ova-pulsed and LPS potentiated DCs or in the presence of ova-pulsed and MPL® immunostimulant-activated DCs and the IL-2 secretory response was measured (FIG. 9a and b). The test population of bone marrow derived DCs were grown for 6 days in the presence of GM-CSF alone or GM-CSF plus increasing concentrations of IL-4 (i.e., 5, 10 and 20 ng/ml, as indicated) prior to antigen and lipid A treatment. The absolute levels of IL-2 response compared to ova-antigen-only DC controls harvested from the same GM-CSF plus IL-4 expansion cultures were determined. The background IL-2 effect for ova-only treated, control (GM-CSF only) 6 day DCs was significant, at approximately 5000 pg/ml (FIG. 9a and b). The IL-2 effect produced by both LPS and MPL® immunostimulant induction of these GM-CSF-only cultured DCs was, however, substantially higher (i.e., 11,000 to 15,000 pg/ml). Comparable to the IFNγ results, the GM-CSF/IL-4 treated, 6 day precultured, DCs showed significantly lower levels of ova-antigen-only induced background IL-2 response. By contrast to the IFNγ data, the GM-CSF/IL-4 precultured DCs showed virtually no ability to respond to either LPS or MPL® immunostimulant stimulation as measured by their low potential to trigger D011.10 T cell IL-2 secretion. The strength of the LPS- and MPL® immunostimulant-induced, antigen-specific, DC-mediated, IL-2 cytokine response for each DC test population was plotted as the % response above its corresponding ova-antigen-only DC background control (FIG. 9c). By contrast to the IFN-γ results, both LPS and MPL® immunostimulant stimulated a stronger DC mediated IL-2 secretory response from D011.10 T cells when “immature” (GM-CSF only cultured) dendritic cells were used. GM-CSF plus IL-4 preculture of DCs produced a DC population which did not promote high levels of IL-2 secretion by ova specific D011.10 T cells.

### Example 6

### LPS Stimulation of 10 Day Murine Bone Marrow Derived DCs Grown in GM-CSF Only

Murine bone marrow derived DCs were typically cultured using a commonly accepted procedure dependent on the combined use of GM-CSF and IL-4 in 6-7 day in vitro cultures. As shown above, these culture conditions produced relatively mature DCs which could still be stimulated by LPS or MPL immunostimulant to obtain enhanced APC function as measured by increased expression of IFN-γ release by D011.10 T cells. A more immature DC phenotype seemed to grow out of GM-CSF only augmented 6 day bone marrow cultures. This type of more immature cell could be further stimulated by antigen and either LPS or MPL® immunostimulant to drive the secretion of IL-2 by ova specific D011.10 T cells. These 6 day DC cultures typically yielded about 5×10⁶ DCs per mouse after 6-7 days of culture. Recently, a 10-12 day DC in vitro culture system in which bone marrow cells are grown in the presence of GM-CSF only has been described (Lutz et al., J. Immunol. Methods 223:77-92 (1999)). Under these culture conditions, the DC yield was reported to be 1-3×10⁸ per mouse and consisted predominantly of immature DCs with a minor mature DC subpopulation. Major modifications were introduced into this culture system. For example, any active depletion of bone marrow cell populations was avoided to eliminate the possible loss of DC precursors. Lower plating density of bone marrow cells was used and the cell culture period was prolonged to 10-12 days. In addition, the GM-CSF dose was reduced from day 8 onward to reduce granulocyte outgrowth contamination. Under such cell culture conditions, the final nonadherent population at day 10-12 was described as being a mixture of both mature and immature DCs. Moreover, further maturation of the DCs was reported to be induced by LPS or TNF-α stimulation for the last 24 hours of culture.

An experiment was carried out using this 10 day DC in vitro system, in order to produce larger numbers of more quiescent, immature DCs which could subsequently be used to more effectively demonstrate the enhanced APC-inductive effects of LPS, MPL® immunostimulant, and synthetic lipid A mimetics. DCs were thus harvested from the 10 day expansion culture system, pulsed with ova for four hours and subsequently cultured with LPS or MPL® immunostimulant for 24 hours. These stimulated DCs were then collected, washed and titered against D011.10 splenic T cells, at a T cell to APC ratios of 4:1 to 128:1, in a 96 well plate format. Culture supernatants were collected after 24-26 hours and tested for IL-2 and IFN-γ. As with the more standard 6 day DC culture system only nonadherent DCs were primarily collected at the end of the 10 day expansion culture for further study. However, in this first 10 day DC experiment, 10 day “mixed” cultures of primarily adherent macrophage and nonadherent DCs were also directly pulsed with ova. LPS or MPL® immunostimulant were then also directly added to these ova-pulsed, 10 day “mixed” cultures for an additional 24 hours prior to harvesting only the nonadherent DCs for use in the D011.10 assay. The stimulation of “mixed” macrophage and DC cultures was carried out in an effort to determine if autologous macrophages might suppress the APC function of DCs harvested from the same culture plate.

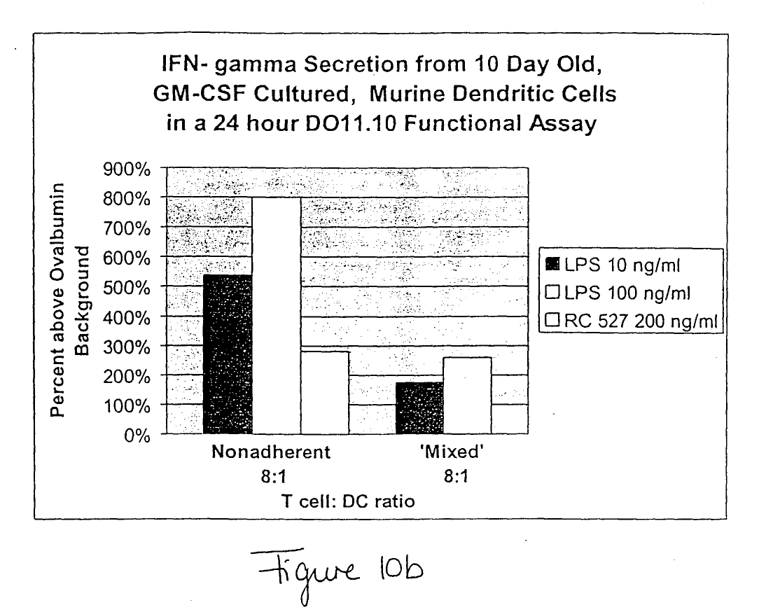

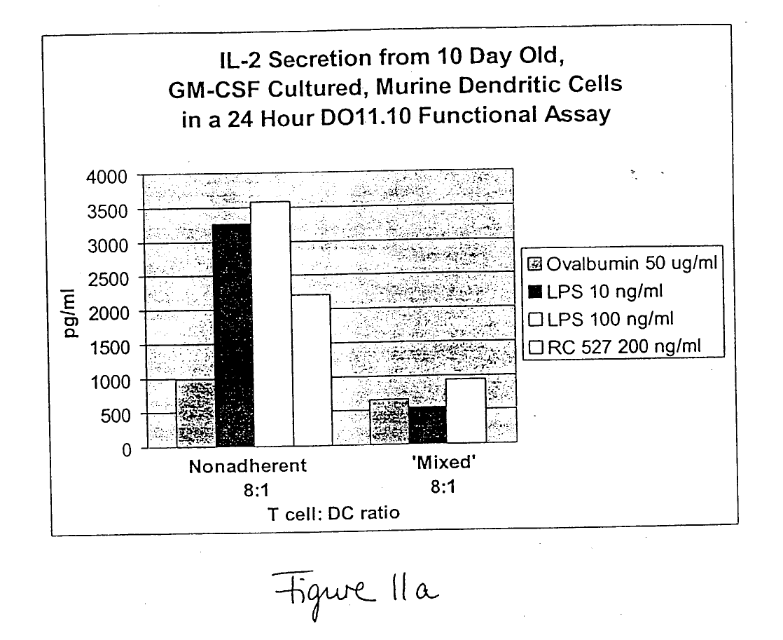

The effect of LPS-stimulated, ova-pulsed, 10 day nonadherent DCs and DCs harvested from “mixed” (macrophage plus DC) induction cultures in triggering IFN-γ release from DC11.10 T cells at a ratio of 8:1 (T cells to DCs) was tested. Overall, the IFNγ effect produced by 10 day (GM-CSF only) nonadherent DCs was weaker in absolute terms compared to the IFN-γ effect produced by 6 day (GM-CSF only) DCs (i.e., ˜2000 pg/ml versus ˜4200 pg/ml; FIG. 10a). However, the ova only DC background control cells from 10 day GM-CSF cultures had a significantly lower background IFN-γ inductive effect (i.e., 350 pg/ml versus 4000 pg/ml), and thus, 10 day (GM-CSF only) LPS-stimulated, ova-pulsed DCs displayed a much higher % above the ova-only background IFNγ effect (FIG. 10b). By contrast, DCs harvested from “mixed” LPS induction cultures (containing both adherent macrophage and nonadherent DCs) showed an overall weaker potential to trigger IFN-γ from D011.10 effector T cells.

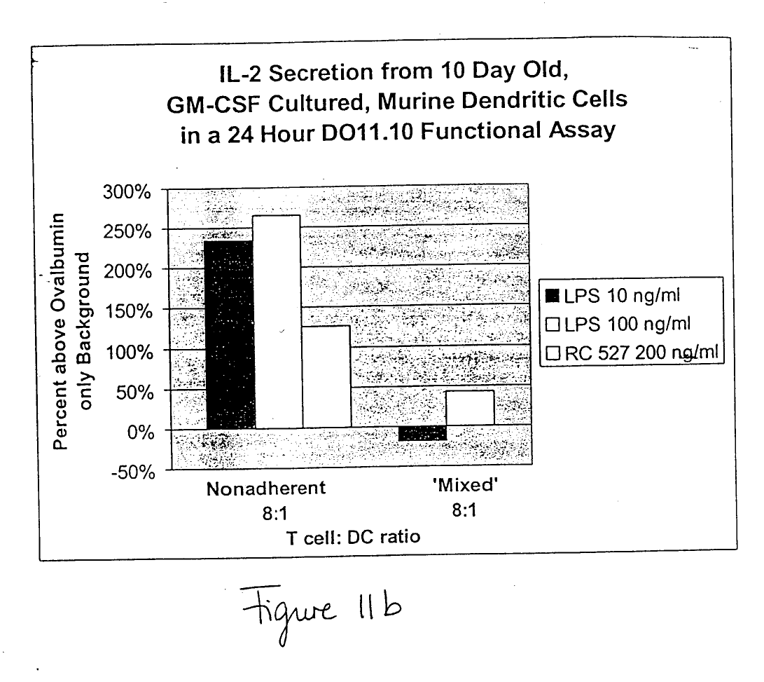

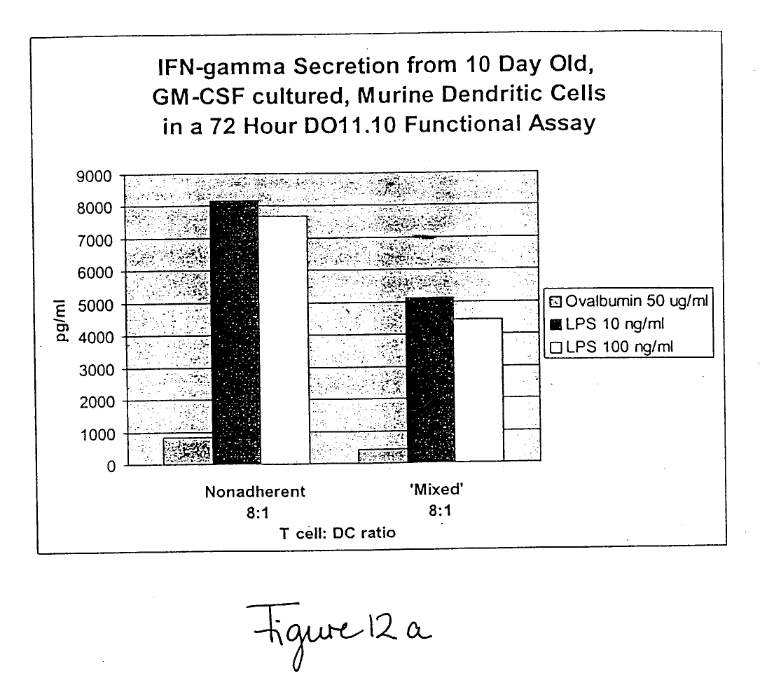

The induction of IL-2 expression by D011.10 cells subsequent to 24 hour in vitro stimulation by LPS-induced, ova-pulsed 10 day DCs precultured in GM-CSF only gave similar results (FIG. 11a and b). Again, the percentage of IL-2 effect above the ova-only DC background values was higher than that seen for 6 day GM-CSF only cultured DCs. However, the absolute level of IL-2 release triggered by LPS-induced 10 day DCs was lower (about 3250 pg/ml) than the IL-2 effect (11,500 pg/ml) observed with 6 day (GM-CSF only) DCs.

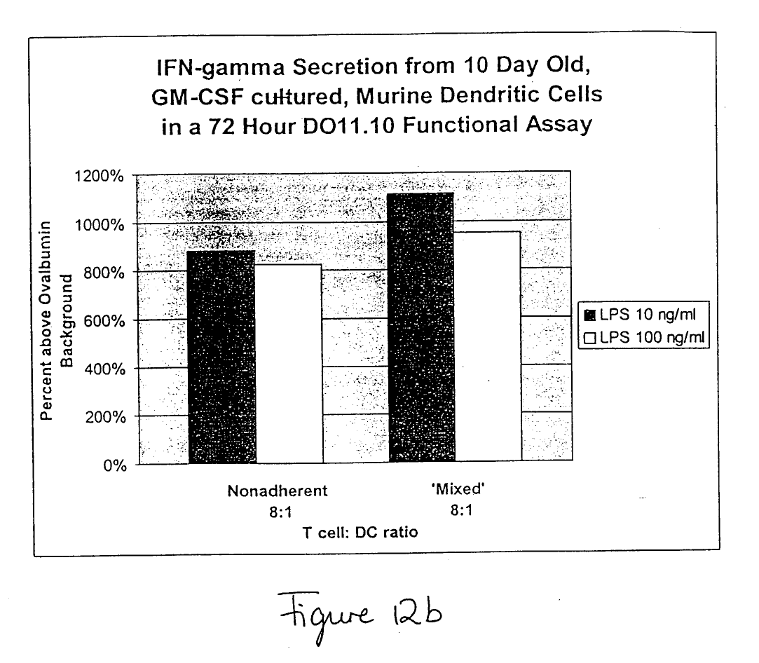

Another set of correlated data for the IFN-γ inductive effects of 10 day GM-CSF only precultured DCs, pulsed with ova and potentiated overnight with LPS were obtained (FIGS. 12a and b). Experiments were carried out with both nonadherent DCs as well as DCs collected from “mixed” LPS induction cultures, but here the supernatants were collected from a 72 hour D011.10 plus DC (8:1 ratio) 96 well plate culture format instead of the 24 hour, 96 well culture format described above. As shown, 72 hour culture supernatants produced significantly higher IFNγ effect with overall IFN-γ levels equal to or higher than those observed using 6 day GM-CSF only precultured, non-adherent DCs. Most significantly, the background IFNγ secretion triggered by DCs pulsed with ova only was demonstrably lower than that observed using 6 day ova-pulsed DC control cells. Thus, the overall LPS stimulation effect compared to the antigen only treated control DC effect was notably higher than that observed with 6 day GM-CSF only precultured DCs.

### Example 7

### Effect of LPS and Synthetic Lipid A Mimetic Stimulation of Cryogenically Preserved Murine Bone Marrow-Derived Dendritic Cells

In order to determine if 10 day GM-CSF precultured DCs could be recovered from cryogenic storage and still retain their functional potential, studies were carried out to determine the functional integrity of LPS- and RC-527-stimulated, cryogenically preserved, 10 day DCs in the D011.10 T cell assay system. Specifically, 10 day GM-CSF-precultured DCs were stored in liquid nitrogen for several weeks. The cells were subsequently thawed and maintained for 24 hours in tissue culture in the presence of GM-CSF. After 24 hours in this “resting” culture, DCs were pulsed with ova for four hours and potentiated with LPS for 24 hours prior to placing them against D011.10 effector T cells at 4:1 and 8:1 ratios.

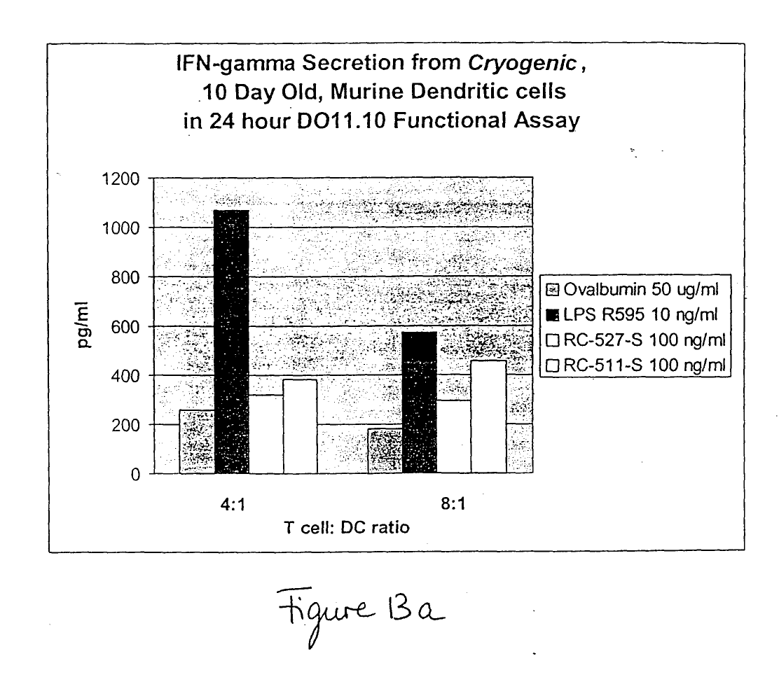

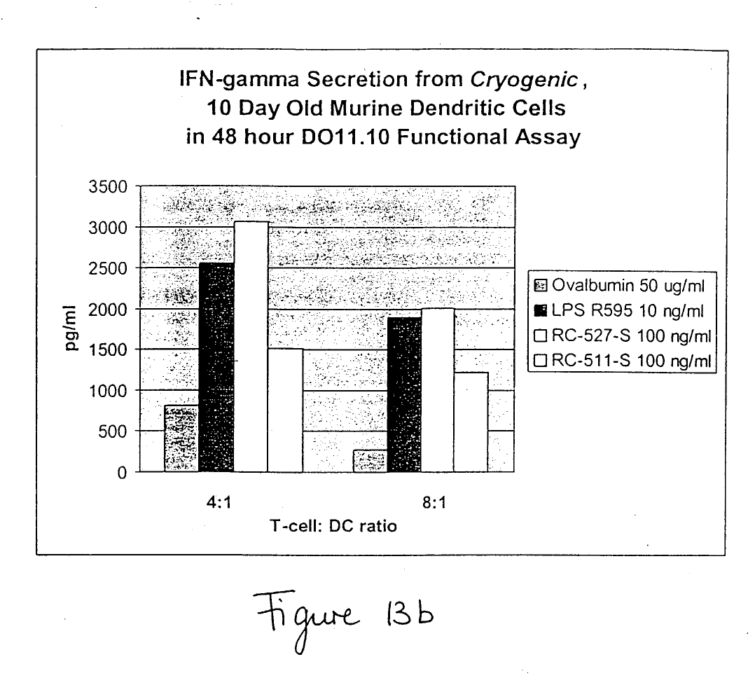

D011.10 effector T cell cultures were maintained for 24 hours and 48 hours, and culture supernatants were collected at both time points. These supernatants were tested for IFN-γ using a commercial ELISA system. The IFNγ response induced either by antigen-pulsed, lipid A stimulated DCs after 24 hours incubation with D011.10 T cells (FIG. 13a) or by LPS and RC-527 stimulated, cryogenically stored, cultured DCs after 48 hours of incubation with D011.10 effector T cells (FIG. 13b) was measured. The LPS and RC-527 stimulated, cryogenically stored, cultured DCs mediated a significantly stronger IFN-γ response after 48 hours of incubation with D011.10 effector T cells. In fact, the 48 hours IFN-γ response triggered by LPS and RC-527-stimulated, cryogenically stored DCs was comparable to the 24 hour IFN-γ response mediated by 10 day GM-CSF precultured DCs harvested directly from the expansion culture system prior to LPS and RC-527 stimulation (FIG. 13c). In this T cell effector assay system, 10 day GM-CSF precultured DCs stored under cryogenic conditions were thus fully capable of supporting an IFNγ response after antigen pulsing and LPS or RC-527 activation. This response was very comparable to the APC effect observed using unfrozen, normal, cultured 10 day DCs.

### Example 8

### Human Dendritic Cell Studies—Measurement of Synthetic Lipid A Mimetic-Induced APC Function by Peripheral Blood-Derived, Cultured Dendritic Cells

The effects of synthetic lipid A mimetics on cultured human DCs derived from CD14+ monocyte precursors collected from peripheral blood were analyzed following the protocol described supra. The ability of cultured CD14+ precursor-derived human DCs to show enhanced APC function after pulsing with the recall antigen, tetanus toxin, and then stimulating the DCs with LPS, MPL® immunostimulant or selected synthetics was analyzed. These cultured antigen-pulsed, lipid A-stimulated DCs were then added to autologous, frozen, peripheral blood T cells collected from the original donor “buffy coat” preparation used to also collect the DC precursor cells. The autologous T cells were stored at −70° C. in liquid nitrogen for 6-7 days, while the DCs were being expanded in vitro with GM-CSF and IL-4. After the DCs were harvested, pulsed with tetanus toxin and treated overnight (i.e., 18-24 hours) with MPL® immunostimulant or with the synthetic mimetics, the T cells were thawed, washed and mixed with the DCs at various T cell:DC ratios, in a 96-well tissue culture plate format. These cultures were incubated for five days and then pulsed with ³H-thymidine (0.5 μci per well) for 18-20 hours. The 96-well plates were harvested on a “MASH” unit, and the incorporated ³H-thymidine was measured by scintillation counting to determine the level of T cell proliferation. T cell proliferation was quantitated as an indication of the strength of the APC function provided by the stimulated DCs. DCs pulsed with tetanus toxin only were used as the background control APC test group.

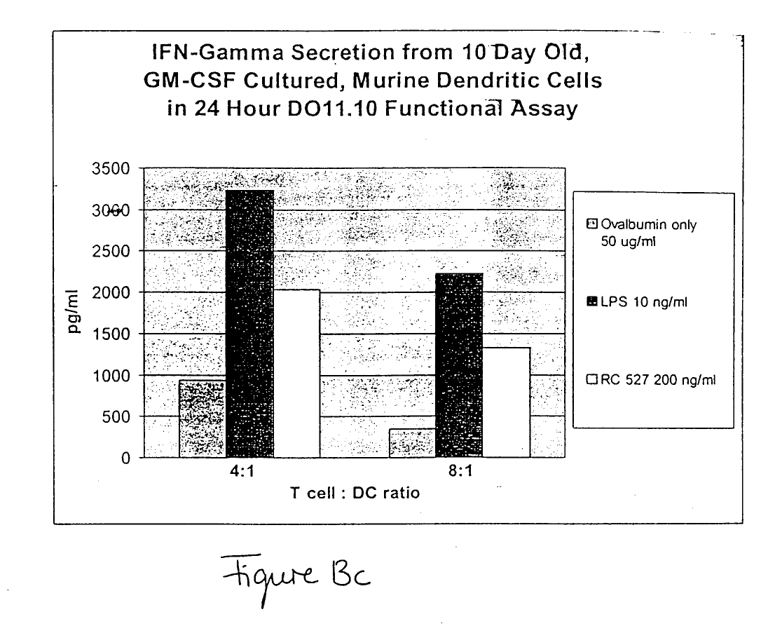

Tetanus toxin-pulsed (four hours), lipid A-stimulated (20 hours), 7 day cultured human DCs were tested for APC function against autologous, cryogenically stored T cells in a 6 day proliferation assay (FIG. 14). The CPM ³H-thymidine response was determined for in vitro cultures of autologous T cells and stimulated DCs where the ratio of T cells to DC was 30:1 (FIG. 14a). As shown, all the DC test groups indicated stimulated significant levels of T cell proliferation that were above that observed for DCs pulsed with the “recall” tetanus antigen alone. Within the context of this single study, the synthetic AGP molecules, RC-527 and RC-544, and the disaccharide glycolipid mimetic, RC-511, all showed the highest levels of DC activation compared to MPL® immunostimulant. Both MPL® immunostimulant and RC-529 were comparable in their effect and quantitatively higher than the background response produced by the antigen-only DC control group. The stimulation index values were determined for each of the test DC groups relative to the background tetanus antigen-only control DC test group (FIG. 14b).

### Example 9

### Human Dendritic Cell Studies—Measurement of Cell Surface Activation Markers after AGP Stimulation

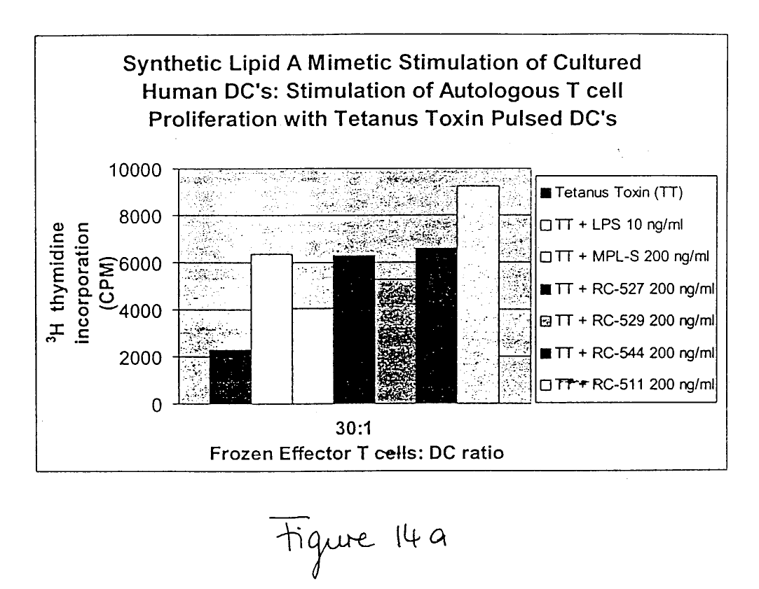

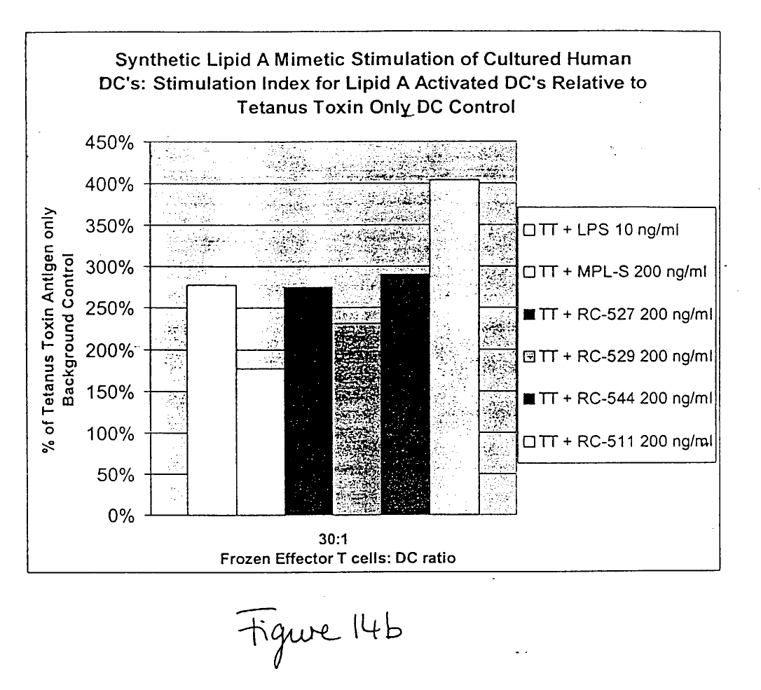

The following example shows a series of titration curves showing the in-vitro dose effect of selected AGPs on the upregulation of various cell surface activation markers on cultured human dendritic cells (DC). Dendritic cells are developed in-vitro from human CD14+ monocyte precursors using standard 7 day GM-CSF (1000 μg/ml) and IL-4 (5004 μg/ml) culture conditions. The cultured DCs are >90% CD11c⁺/HLA-DR⁺/lineage⁻ by flow cytometry analysis. FIGS. 15-18 show the results of flow cytometry analysis of gated CD11c⁺/HLA-DR⁺/lineage⁻ human DCs after they were restimulated in-vitro for 72 hours in the presence of selected AGPs at a titration range of 0.1 μg/ml-2 μg/ml. Data is expressed as the percent of gated DCs which are positive for the indicated marker (y-axis) versus the logo₁₀ of the AGP or lipid A concentration. R595 LPS was used in each induction at 10 ng/ml as a positive control—the negative control was unstimulated human dendritic cells cultured for 72 hours in the same medium conditions used for AGP stimulation. Data was analyzed using non-linear regression analysis—all data was modeled using a 3rd order polynomial to determiine the best curve fit.

FIG. 15: RC527 stimulates the highest level of increased cell surface expression of the costimulatory molecule, CD80. The RC527 effect is sustained over the entire titration range as shown, and is almost equal to the response generated by the positive control, R595 LPS at 10 ng/ml. The dose effect of de-phosphorylated lipid A from Avanti Polar Lipids (Alabaster, Ala.) and RC 590 are very comparable and seem to plateau at 1-2 μg/ml. MPL-AF and RC524 are also similar in their effect across the titration range and appear to be still rising at the high dose concentration of 2 μg/ml. RC529 produces the lowest overall increase in CD80 expression and the effect appears to plateau at 1 μg/ml. In this and all subsequent experiments Corixa AGPs, R595 LPS, and MPL were all used as stock AF formulations provided by Corixa-Montana while the de-phosphorylated lipid A (Avanti Lipid A) was used as a 10% ethanol-in-water formulation.

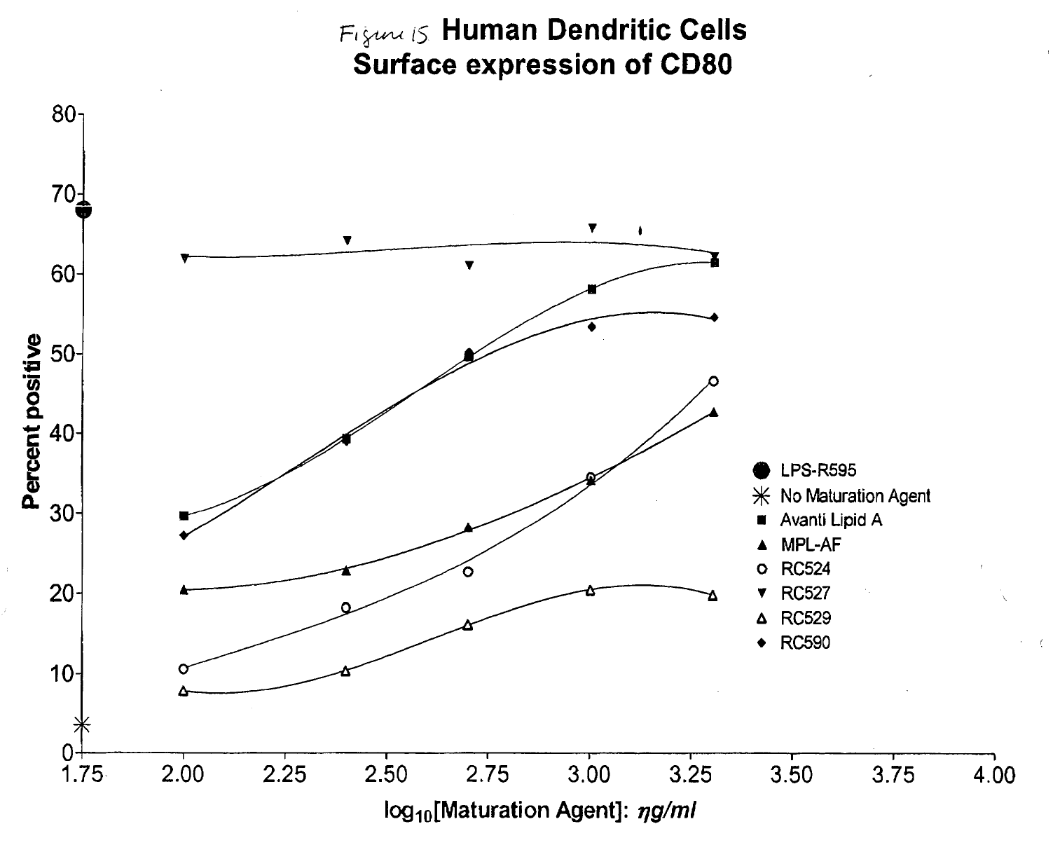

FIG. 16: Again, RC527 stimulated the highest DC expression levels of the maturation molecule CD83, and this level of cell surface expression was sustained over the titration range of the AGP. Similarly, Avanti Polar Lipid and RC590 again stimulated very comparable, high CD83 response which seemed to plateau at 1-2 μg/ml. MPL-AF stimulated a lower overall dose titration response which appeared to be still increasing at 2 μg/ml. RC524 did not stimulate a CD83 response much above the negative control background until a concentration of 1 μg/ml and the effect seemed to continue to increase at the highest concentration. RC529 stimulated a weak overall increase in CD83 expression across the entire AGP dose range which was slightly above the CD83 expression measured in the non-stimulated, negative control DC culture.

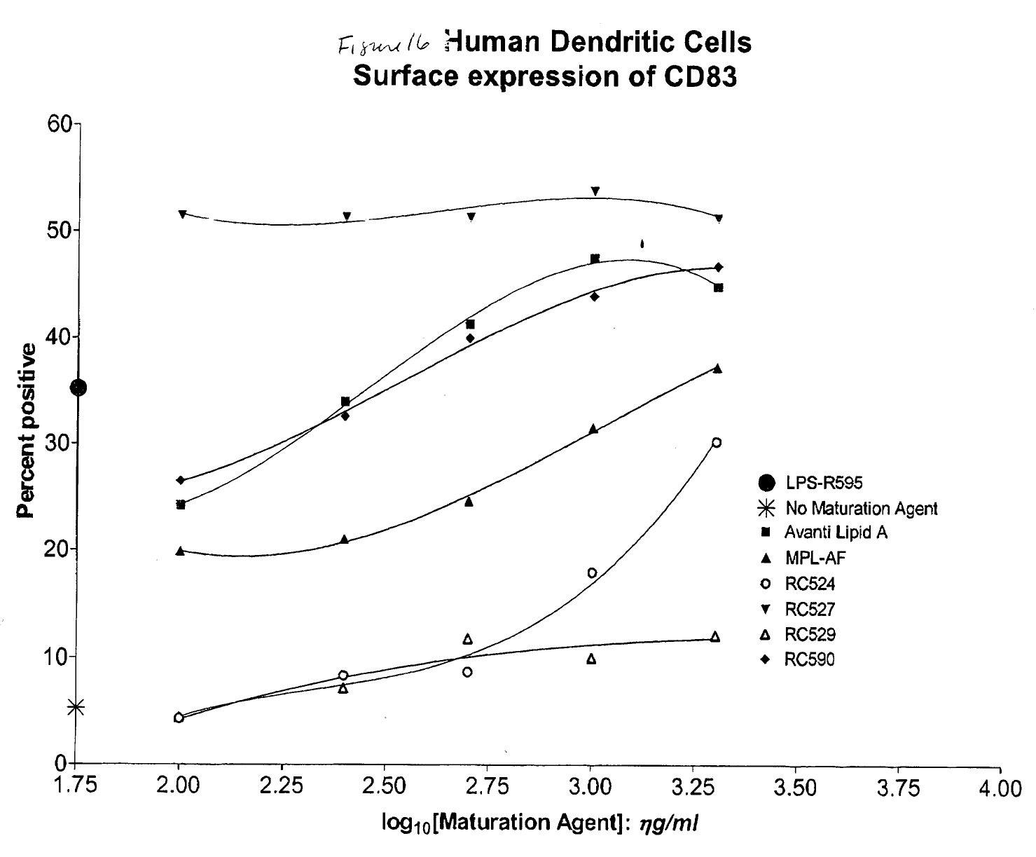

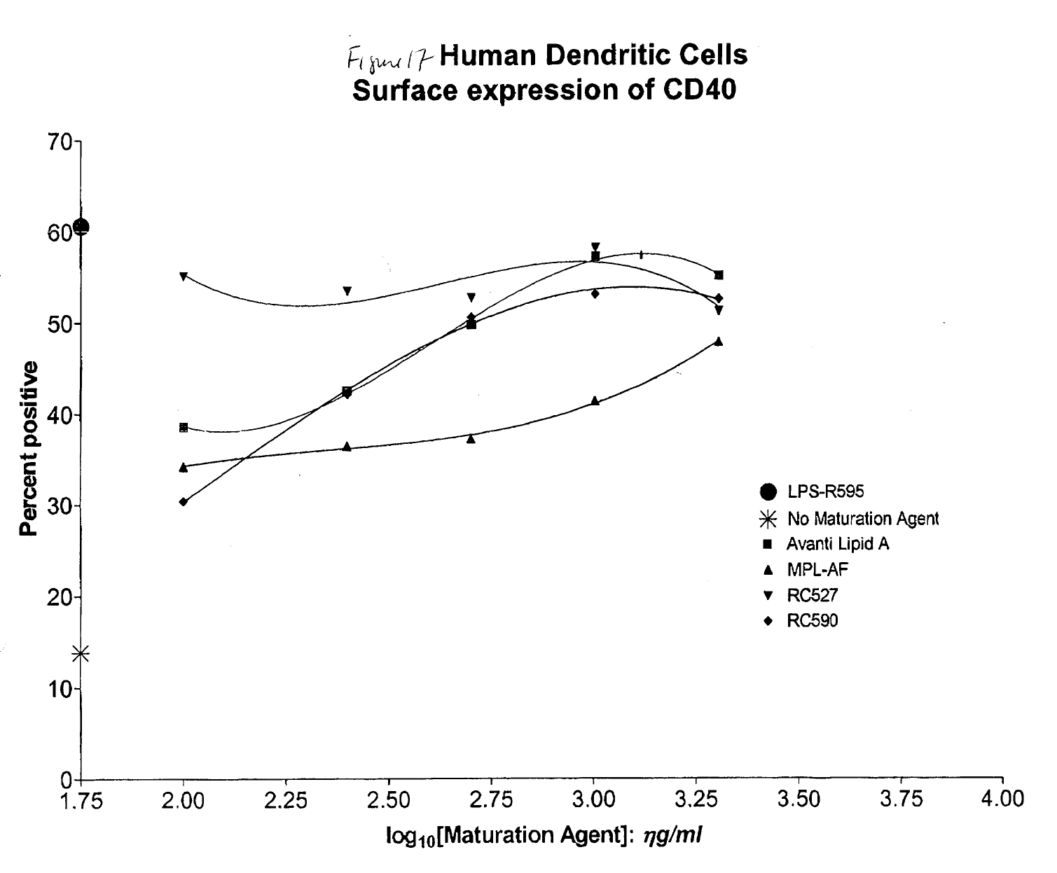

FIG. 17: RC527 stimulated the highest elevated cell surface expression of the important DC co-stimulatory molecule, CD40. Again, this effect was sustained over the entire AGP dose range and was almost equal to the R595 LPS-induced response at 10 ng/ml. Both Avanti Lipid A and RC590 produced very similar levels of CD40 expression—this effect was essentially equal to the response generated by RC527 at the higher AGP concentrations of 1-2 μg/ml where the response plateaued for both RC590 and Avanti Lipid A. MPL-AF demonstrated very effective stimulation of CD40 expression which was still increasing at the highest dose of 2 μg/ml. In this analysis we did not compare the effects of RC524 and RC529 for the stimulation of CD40 expression.

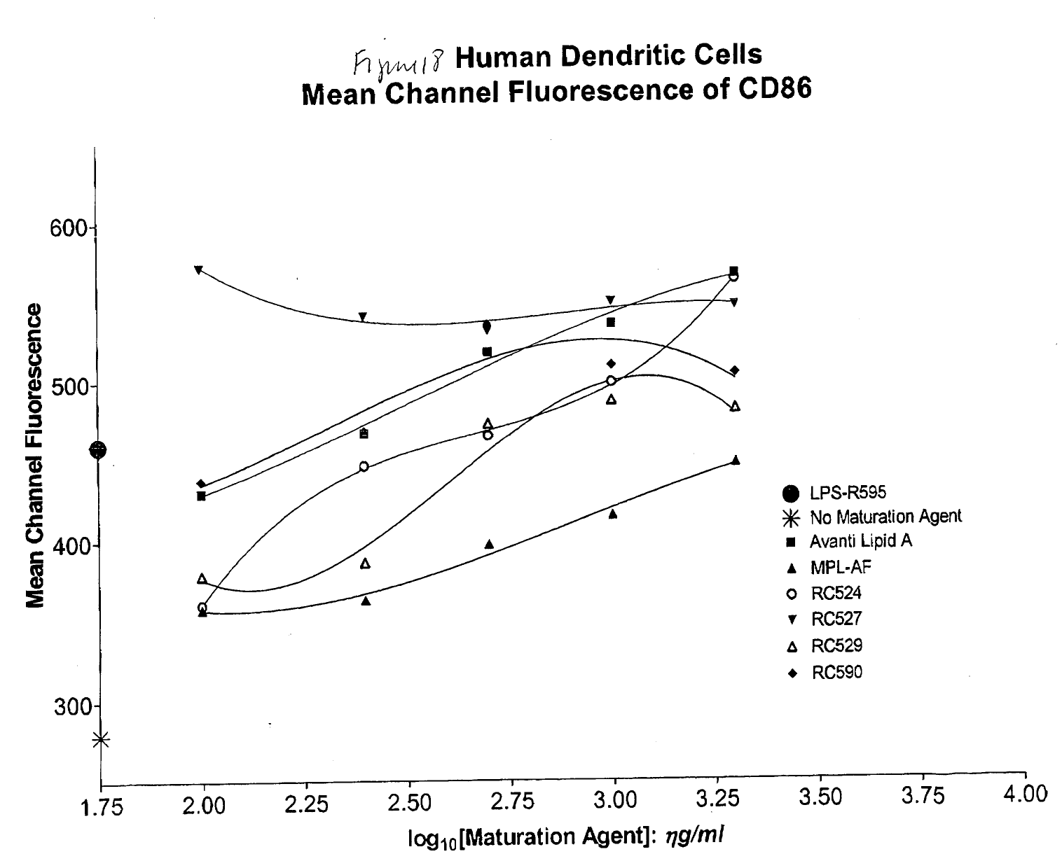

FIG. 18: The co-stimulatory molecule, CD86, is frequently expressed constitutively on cultured DCs from individual donors. The donor DCs used in this study were >85% CD86⁺ after standard GM-CSF/IL-4 culture, and did not increase with AGP or Lipid A restimulation. However, the average cell surface density of CD86 expression for stimulated DCs did increase (expressed as the mean channel fluorescence value) with increased concentrations of Lipid A or AGP molecules. The mean channel fluorescence (MCF) values are essentially measurements of the average cell surface epitope density, or copy number for the CD86 molecule. As shown in FIG. 18—RC527 sustains the highest stimulation of CD86 expression over most of the titration range. The effect induced by Avanti-Lipid A and RC590 are again very comparable over most of the dose range—interestingly the RC590 effect seems to plateau or even drop at 1 μg/ml while the stimulated expression of CD86 is still increasing at 1-2 μg/ml of Avanti Lipid A. RC524 and RC529 stimulate similar increases in CD86 expression over the lower end of the titration—the RC529 effect appears to maximize at 1 μg/ml while that of RC524 is still rising at the highest concentration of 2 μg/ml. MPL-AF shows the lowest overall stimulated effect—interestingly all of the molecular species compared in this analysis show the ability to stimulate higher CD86 expression at 500 ng/ml than does the LPS positive control. Only MPL-AF fails to stimulate CD86 expression to levels equal to those induced by 10 ng/ml of R595 LPS.

Overall the data from this single analysis of DCs cultured from an individual normal donor indicate that RC527 stimulates the strongest induction of DC maturation and co-stimulatory molecule expression of all the AGPs tested. This effect is frequently equal to or greater than the response stimulated by the positive control molecule, R595 LPS (10 ng/ml), and in this study is usually already maximized at 100 ng/ml concentration. RC590 also produces strong DC inductive effects—especially at concentrations ≧1 μg/ml, and these stimulation responses are very comparable to those induced by the commercially available Avanti Lipid A. MPL-AF demonstrates good stimulation of CD40, CD83, CD80 expression which does not appear to be maximal even at concentrations of 1-2 μg/ml. At concentrations of 1-2 μg/ml RC524 stimulates comparable effects to those of MPL-AF at the same dose. RC529, by comparison, stimulates very low to marginally positive CD80 and CD83 expression while sustaining strong upregulation of MCF for CD86 expression a concentration of 1-2 μg/ml.

From the foregoing, it will be appreciated that, although specific embodiments of the invention have been described herein for the purpose of illustration, various modifications may be made without deviating from the spirit and scope of the invention.

What is claimed is:

1. A method of inducing maturation of dendritic cells ex vivo, the method comprising the steps of:
(i) incubating a culture of cells comprising dendritic cell progenitors with at least one cytokine or chemokine that promotes differentiation of dendritic cell progenitors;
(ii) pulsing the cells with antigen; and
(iii) stimulating the cells with a compound of Formula I below: 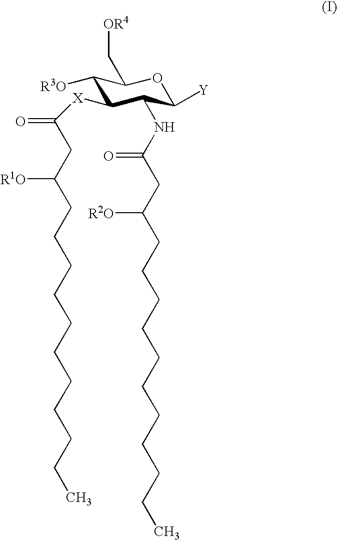
and pharmaceutically acceptable salts thereof, wherein X is —O— or —NH—; R¹ and R² are each independently a (C₂-C₂₄)acyl group, including saturated, unsaturated, substituted, unsubstituted, straight, and branched acyl groups; R³ is —H or —PO₃R¹²R¹³, wherein R¹² and R¹³ are each independently —H or (C₁-C₄)alkyl; R⁴ is —H, —CH₃ or —PO₃R¹⁴R¹⁵, wherein R¹⁴ and R¹⁵ are each independently selected from —H and (C₁-C₄)alkyl; and Y is a radical selected from the formulae: 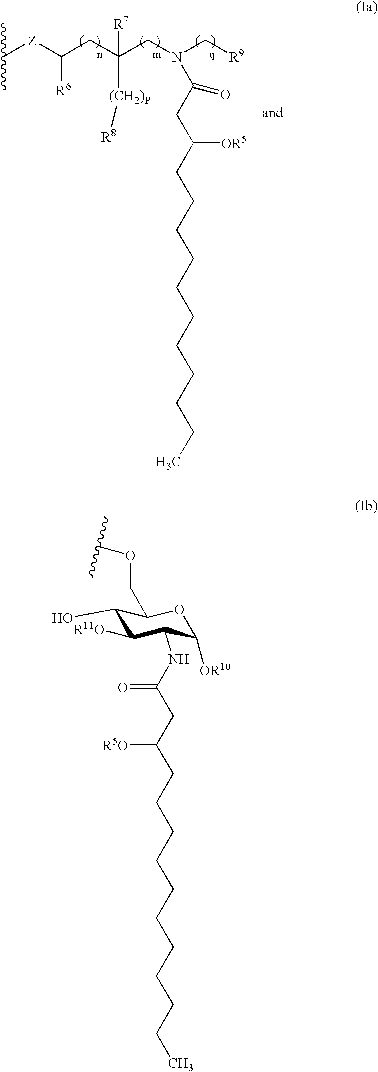
wherein the subscripts n, m, p and q are each independently an integer of from 0 to 6; R⁵ is H or a (C₂-C₂₄)acyl group including saturated, unsaturated, substituted, unsubstituted, straight, and branched acyl groups; R⁶ and R⁷ are independently selected from H and CH₃; R⁸ and R⁹ are independently selected from H, OH, (C₁-C₄)alkoxy, —PO₃H₂, —OPO₃H₂, —SO₃H, —OSO₃H, —NR¹⁶R¹⁷, —SR¹⁶, —CN, —NO₂, —CHO, —CO₂R¹⁶, and —CONR¹⁶R¹⁷, wherein R¹⁶ and R¹⁷ are each independently selected from H and (C₁-C₄)alkyl; R¹⁰ is selected from H, CH₃, —PO₃H₂, ω-phosphonoxy(C₂-C₂₄)alkyl, and ω-carboxy(C₁-C₂₄)alkyl; R¹¹ is H or a (C₂-C₂₄)acyl group including saturated, unsaturated, substituted, unsubstituted, straight, and branched acyl groups; and Z is —O— or —S—; with the proviso that when R³ is —PO₃R¹²R¹³, R⁴ is other than —PO₃R¹⁴R¹⁵;
thereby inducing maturation of dendritic cells.

2. The method of claim 1, wherein the cell is a human cell.

3. The method of claim 1, wherein the cytokine is selected from the group consisting of GM-CSF, IL-4, and TGF-β.

4. The method of claim 1, wherein the cytokine is GM-CSF.

5. The method of claim 4, wherein the GM-CSF is exogenously added to the culture.

6. The method of claim 5, wherein the cells are incubated with exogenously added GM-CSF for 5-7 days.

7. The method of claim 5, wherein the cells are incubated with exogenously added GM-CSF for 10-12 days.

8. The method of claim 4, wherein the cells are further incubated with IL-4.

9. The method of claim 4, wherein the cells are further incubated with TGF-β.

10. The method of claim 4, wherein the cells are further incubated with TGF-β and IL-4.

11. The method of claim 1, wherein the cells are pulsed with antigen after stimulation of the cells with the compound of formula I.

12. The method of claim 1, wherein the cells are pulsed with antigen before stimulation of the cells with the compound of formula I.

13. The method of claim 1, wherein the culture of cells is isolated from bone marrow.

14. The method of claim 1, wherein the culture of cells is isolated from PBMC.

15. The method of claim 14, wherein the culture of cells is isolated from CD14+ or CD34+ PBMC.

16. The method of claim 1, further comprising the step of cryogenically storing the cells.

17. The method of claim 1, wherein the antigen is selected from the group consisting of a cancer antigen, a viral antigen, a bacterial antigen, and a parasitic antigen.

18. The method of claim 17, wherein the antigen is from a Mycobacterium sp., Chlamydia sp., Leishmania sp., Trypanosoma sp., Plasmodium sp., or a Candida sp.

19. The method of claim 17, wherein the antigen is from Mycobacterium tuberculosis.

20. The method of claim 17, wherein the antigen is from Trypanosoma cruzii.

21. The method of claim 1, wherein the antigen is associated with an autoimmune disorder.

22. The method of claim 1, wherein R₁═R₂═R₅═N—C₁₃H₂₇CO, X═Y═O, N=M=P=Q=0, R₆═R₇═R₈═R₉═R₄═H, and R₃═PO₃H₂.

23. The method of claim 1, wherein R₁═R₂═R₅═N—C₁₁H₂₃CO, X═Y═O, N=M=Q=0, P=1, R₄═R₆═R₇═R₉═H, R₈═OH, and R₃═PO₃H₂.

24. The method of claim 1, wherein R₁═R₂═R₅═N—C₉H₁₉CO, X═Y═O, N=M=Q=P=0, R₄═R₆═R₇═R₉═H, R₈═CO₂H, and R₃═PO₃H₂.

25. A method of eliciting an immune response in a mammal, the method comprising the steps of:
(i) isolating a culture of cells from the mammal, wherein the cells comprise dendritic progenitors;
(ii) incubating the culture of cells with at least one cytokine or chemokine that promotes differentiation of dendritic cell progenitors;
(iii) pulsing the cells with an antigen;
(iv) stimulating the cells with a compound of Formula I below: 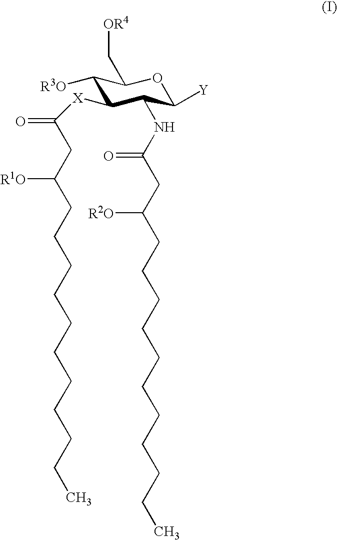
and pharmaceutically acceptable salts thereof, wherein X is —O— or —NH—; R¹ and R² are each independently a (C₂-C₂₄)acyl group, including saturated, unsaturated, substituted, unsubstituted, straight, and branched acyl groups; R³ is —H or —PO₃R¹²R¹³, wherein R¹² and R¹³ are each independently —H or (C₁-C₄)alkyl; R⁴ is —H, —CH₃ or —PO₃R¹⁴R¹⁵, wherein R¹⁴ and R¹⁵ are each independently selected from —H and (C₁-C₄)alkyl; and Y is a radical selected from the formulae: 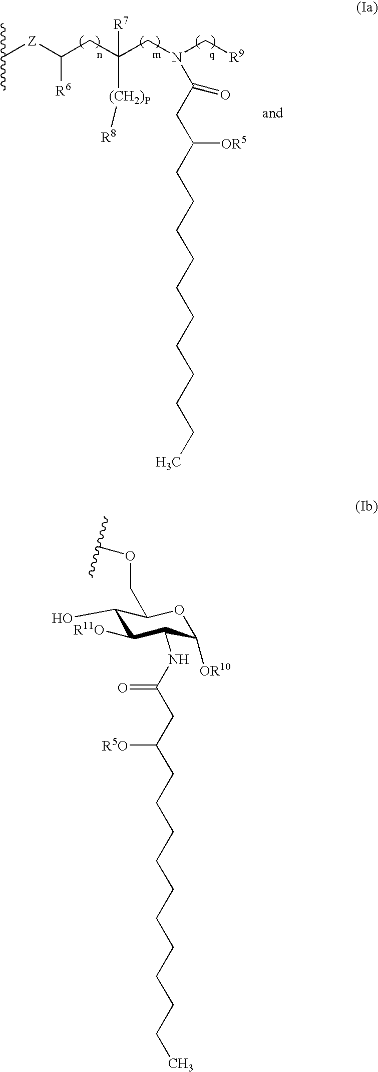
wherein the subscripts n, m, p and q are each independently an integer of from 0 to 6; R⁵ is H or a (C₂-C₂₄)acyl group including saturated, unsaturated, substituted, unsubstituted, straight, and branched acyl groups; R⁶ and R⁷ are independently selected from H and CH₃; R⁸ and R⁹ are independently selected from H, OH, (C₁-C₄)alkoxy, —PO₃H₂, —OPO₃H₂, —SO₃H, —OSO₃H, —NR¹⁶R¹⁷, —SR¹⁶, —CN, —NO₂, —CHO, —CO₂R¹⁶, and —CONR¹⁶R¹⁷, wherein R¹⁶ and R¹⁷ are each independently selected from H and (C₁-C₄)alkyl; R¹⁰ is selected from H, CH₃, —PO₃H₂, ω-phosphonoxy(C₂-C₂₄)alkyl, and ω-carboxy(C₁-C₂₄)alkyl; R¹¹ is H or a (C₂-C₂₄)acyl group including saturated, unsaturated, substituted, unsubstituted, straight, and branched acyl groups; and Z is —O— or —S—; with the proviso that when R³ is —PO₃R¹²R¹³, R⁴ is other than —PO₃R¹⁴R¹⁵.
thereby inducing maturation of dendritic cells that present epitopes of the selected antigen; and
(iv) administering an immunogenically effective amount of the cells to the mammal, thereby eliciting an immune response to the selected antigen.

26. The method of claim 25, wherein the mammal is a human.

27. The method of claim 25, wherein the cell is a human cell.

28. The method of claim 25, wherein the cells are pulsed with antigen after stimulation of the cells with the compound of formula I.

29. The method of claim 25, wherein the cells are pulsed with antigen before stimulation of the cells with the compound of formula I.

30. The method of claim 25, wherein the cytokine is selected from the group consisting of GM-CSF, IL-4, or TGF-β.

31. The method of claim 25, wherein the cytokine is GM-CSF.

32. The method of claim 31, wherein the GM-CSF is exogenously added.

33. The method of claim 32, wherein the cells are incubated with exogenously added GM-CSF for 5-7 days.

34. The method of claim 32, wherein the cells are incubated with exogenously added GM-CSF for 10-12 days.

35. The method of claim 31, wherein the cells are further incubated with IL-4.

36. The method of claim 31, wherein the cells are further incubated with TGF-β.

37. The method of claim 31, wherein the cells are further incubated with TGF-β and IL-4.

38. The method of claim 25, wherein the culture of cells is isolated from bone marrow.

39. The method of claim 25, wherein the culture of cells is isolated from PBMC.

40. The method of claim 39, wherein the culture of cells is isolated from C14+ or CD34+ PBMC.

41. The method of claim 25, wherein the antigen is selected from the group consisting of a cancer antigen, a viral antigen, a bacterial antigen, and a parasitic antigen.

42. The method of claim 41, wherein the antigen is from Mycobacterium sp., Chlamydia sp., Leishmania sp., Trypanosoma sp., Plasmodium sp., or a Candida sp.

43. The method of claim 41, wherein the antigen is from Mycobacterium tuberculosis.

44. The method of claim 41, wherein the antigen is from Trypanosoma cruzii.

45. The method of claim 25, wherein the antigen is associated with an autoimmune disorder.

46. The method of claim 25, wherein R₁═R₂═R₅═N—C₁₃H₂₇CO, X═Y═O, N=M=P=Q=0, R₆═R₇═R₈═R₉═R₄═H, and R₃═PO₃H₂.

47. The method of claim 25, wherein R₁═R₂═R₅═N—C₁₁H₂₃CO, X═Y═O, N=M=Q=0, P=1, R₄═R₆═R₇═R₉═H, R₈═OH, and R₃═PO₃H₂.

48. The method of claim 25, wherein R₁═R₂═R₅═N—C₉H₁₉CO, X═Y═O, N=M=Q=P=0, R₄═R₆═R₇═R₉═H, R₈═CO₂H, and R₃═PO₃H₂.
